(19) United States
(12) Patent Application Publication
(10) Pub. No.: US 2024/0082958 A1
(43) Pub. Date: Mar. 14, 2024

(54) LASER ETCHED CAPSULES AND METHODS OF MAKING THEM

(71) Applicant: Bayer HealthCare LLC, Whippany, NJ (US)

(72) Inventors: Debanjan Das, Morristown, NJ (US); Reinhard Walter, Morristown, NJ (US); Javier Zumeta Perez, Alcala de Henares (ES); Alberto Prior Cabanillas, Colmenar Viejo (ES); Gerard Meisel, Budd Lake, NJ (US); Carlos Figueroa, Boonton, NJ (US); Mark David Ford, Sicklerville, NJ (US); Roland Norman Thurston, Jr., Sewell, NJ (US)

(73) Assignees: Bayer HealthCare LLC, Whippany, NJ (US); Ackley Machine Corporation, Moorestown, NJ (US)

(21) Appl. No.: 17/767,840

(22) PCT Filed: Oct. 8, 2020
(86) PCT No.: PCT/US2020/054870

§ 371 Date: Apr. 8, 2022

Related U.S. Application Data
(60) Provisional application No. 62/912,363, filed on Oct. 8, 2019.

(51) Int. Cl. B23K 26/364

(52) U.S. Cl. CPC B23K 26/364; B23K 2101/04

(57) ABSTRACT

The present disclosure is directed to capsules, for example, softgel capsules for oral administration that have on their surface laser etching that penetrates at least partially into the surface of the capsule, reduces the structural integrity of the capsule while preventing exposure of the interior contents to the outside environment, and increases the rate of release of active ingredients contained within.

### TECHNICAL FIELD

The present disclosure is directed to laser etched capsules, for example, softgel capsules for oral administration that contain pharmaceutical ingredients. The capsules have etching that penetrates only partially through surface of the capsule and does not penetrate the capsule or expose the encapsulated contents to the exterior environment. This laser etching provides the benefit of facilitating capsule disintegration and pharmaceutical release after capsule ingestion without undermining the structural integrity of the capsule during manufacturing, storage, or administration. Also disclosed are devices and methods for using the devices and methods for manufacturing such capsules that optimize ablation of the capsule surface and the formation of structurally sound capsules with faster drug release profiles.

### BACKGROUND

One form of oral pharmaceutical delivery is through the use of capsules that contain one or more active ingredients along with different types of excipients in a formulation designed to release active ingredients in the gastrointestinal (GI) tract for absorption and delivery to systemic circulation. Traditionally, the outer shell (the “shell”) of a “gel” capsule is prepared from gelatin, plasticizer(s) and water and can be manufactured in two different forms: “hard gelatin” capsules and “soft gelatin” capsules or “softgels.”

Hard gelatin capsules are typically used to encapsulate solid materials or products that are encapsulated as microparticles containing disintegrants and other excipients to expedite drug delivery. Hard gelatin capsules are typically formed by combining two separate parts fitted together to form a complete capsule, i.e. the “cap” and “body.” These hard shell capsules are not sealed, and, thus, are unsuitable for encapsulation of liquid or gel materials. They also do not prevent exposure of the enclosed active ingredients to air and oxidation.

Softgels, by contrast, can be used to encapsulate drugs that are incorporated into solid, semi-solid or liquid formulations. Softgels offer many advantages over other dosage forms. For example, softgels have the advantage of being sealed and can, therefore, protect the ingredients from exposure to air and oxidation, unlike hard gels, which are not sealed. Softgel capsules are also easy to swallow, can mask the taste and odor of unpleasant ingredients, present an attractive appearance, are digested quickly in the gastrointestinal tract, and can be formulated in a wide variety of colors, shapes, sizes and compounds. In addition, softgel capsules are particularly well-suited for use in quick or rapid release formulations. The liquid or gelatin formulations, which are possible to include in softgels, can contain the active pharmaceutical ingredient (API) in a partially or completely solubilized state. As a result, APIs in softgel formulations typically solubilize and are absorbed faster than those contained in the microparticles or microcapsules incorporated into hard gel capsules, thereby leading to a formulation with a more rapid release profile.

Drug release from a soft gelatin capsule shell is largely dependent upon rupture of gelatin followed by disintegration of the gelatin shell, thereby releasing the drug(s) loaded inside the capsule. The time required by the gelatin to dissolve in the stomach acidic system can vary from 20-60 minutes based on type of the gelatin shell, the nature of the fill, the type of drug(s) molecule(s) and other factors. Since bioavailability of drug molecule(s) is a function of drug(s) release from the capsules, the rupture and disintegration of the gelatin shell becomes a rate limiting factor affecting the onset of therapeutic action. Any rapid drug action by softgel capsules, thus, depends on rapid rupture of gelatin shells and rapid release of the capsule contents.

The literature discloses methods for altering the rate of drug release using a variety of techniques that alter the surface of the capsule or tablet by penetrating the outermost surface to expose the core or an intermediate layer to the external environment. For example, U.S. Pat. No. 5,873,793 discloses the use of pre-drilled holes in hard gel capsules that penetrate the gel surface, facilitate liquid access into the interior of the capsule, and accelerate drug release. U.S. Pat. No. 7,404,708, teaches a machine and method for generating similar tablets. U.S. Pub. No. 20110200537, for example, discloses the use of lasers to drill completely through an outer layer of a capsule and expose the core or an intermediate layer. U.S. Pat. No. 9,149,438, by contrast, discloses a tablet formulation that contains a polymeric sub-coating over the entire tablet with holes that penetrate the subcoating to expose the core of the tablet and with additional coatings on other capsule surface areas. U.S. Pub. No. 20160051479 also discloses outer coatings, in this case gelatinous, with openings that provide contact between the exterior of the coating and the interior contents. U.S. Pat. No. 9,579,289, discloses semipermeable membranes that enclose API but that also have pre-formed passageways to alter drug release kinetics. U.S. Pub. No. 20060177507A1 also discloses controlled release devices for drug delivery that that expose the capsule core to the exterior environment through preformed holes. And U.S. Pat. No. 6,004,582 discloses a multi-layered osmotic device comprising a core surrounded by a semipermeable membrane having a preformed hole in it.

The literature also discloses the use of lasers to etch capsules, not to alter drug release profile, but rather to mark, texturize, colorize, or identify the capsules. For example, U.S. Pub. No. 20130244002 and PCT Pub. No. WO2011011333 disclose the use of lasers to mark pharmaceuticals to prevent counterfeiting. U.S. Pub. Nos. 20090304601 and 20100303735 also teach methods of using lasers to mark capsules for identification purposes.

The literature does not, however, disclose capsules having laser etching that removes a portion of the surface, that does not penetrate to the core of the capsule and expose the ingredients within, but that still facilitates API release through accelerated rupture and disintegration of the capsule in the GI.

### SUMMARY OF THE DISCLOSURE

The present disclosure is generally related to softgel capsules that maintain their structural integrity during manufacturing, storage, and delivery, but that also achieve a faster rupture rate and API release in the digestive tract. The present disclosure provides these characteristics through laser etched furrows or channels situated on the surface of the capsules. These furrows or channels effectively increase the surface area of the softgel capsule exterior exposed to liquid, decrease the thickness of the capsule wall and modify the structural integrity of the capsule at the site of the furrow, and promote water entrapment and eddying at the etching site. In combination, these traits result in increased hydrostatic pressure in the vicinity of the etching site and accelerate rupture of the softgel capsule and release of the enclosed APIs as compared to softgels that lack such etching.

The disclosure relates to laser etched capsules, for example, softgel capsules for oral administration that contain pharmaceutical ingredients, as well as devices and methods for making the same. In some embodiments, the disclosure provides a capsule comprising at least one laser etched channel positioned on an outer surface of the capsule, wherein the at least one channel penetrates at least partially into the surface of the capsule and reduces a structural integrity of the capsule, while preventing exposure of interior contents of the capsule to the outside environment and facilitating release of the interior contents of the capsule. In some embodiments, the at least one laser etched channel facilitates disintegration of the capsule by increasing capsule surface area, facilitating erosion, and reducing capsule thickness at a site of the at least one laser etched channel. In some embodiments, the at least one laser etched channel facilitates release of the interior contents of the capsule through an increase in the rupture rate of the capsule. In some embodiments, the capsule is an oral capsule. In some embodiments, the capsule is a softgel capsule.

The disclosure also relates to a capsule comprising at least one laser etched channel situated on an outer surface of the capsule, wherein the at least one laser etched channel penetrates at least partially into the surface of the capsule while maintaining a structural integrity of the capsule and preventing exposure of the interior contents of the capsule to the outside environment and facilitating the rate of release of the internal contents of the capsule, wherein the at least one laser etched channel provides a localized area of modified thickness and structural integrity at a site of the at least one laser etched channel.

The disclosure further relates to a softgel capsule that contains at least one active pharmaceutical ingredient (API), the capsule comprising at least one laser etched channel situated on an outer surface of the capsule, wherein the at least one laser etched channel penetrates at least partially into the outer surface of the capsule while preventing exposure of the least one active pharmaceutical ingredient to the outside environment and increasing the rate of release in the GI tract of the at least one API as compared to the rate of release in the GI tract of the at least one API of a softgel capsule that has a similar composition but that lacks laser etching.

The disclosure also relates to a method of making an etched capsule, the method comprising ablating a portion of an outer surface of the capsule with at least one laser beam to form at least one laser etching on the surface of the capsule, wherein the at least one laser etching penetrates at least partially into the surface of the capsule, and wherein the at least one laser etching provides a localized area of reduced thickness and reduced structural integrity as compared to a capsule free of laser etching.

The disclosure further relates to a system comprising: (i) a controller; (ii) a device comprising a laser and a processor; and (iii) a non-transitory computer-readable storage medium in operable communication with the controller and the processor, wherein the non-transitory computer-readable storage medium has instructions stored thereon for execution on the device and wherein the processor implements a method comprising: (a) activating a laser positioned as a distance from a capsule; (b) lasing a capsule disclosed herein along a first predetermined path for a time period sufficient to create an etching at a first predetermined depth; (c) pausing the lasing from about 0.1 to about 1 second; and (d) lasing the capsule disclosed herein along a second predetermined path for a time sufficient to create a second etching beginning at least partially at the first predetermined depth for a time period sufficient to create an etching at a second predetermined depth.

The disclosure also relates to a non-transitory computer-readable storage medium having instructions stored thereon for execution of a method comprising: (a) activating a laser positioned as a distance from a capsule; (b) ablating a capsule disclosed herein along a first predetermined path for a time period sufficient to create an etching at a first predetermined depth; (c) pausing the lasing from about 0.1 to about 1 second; (d) ablating the capsule disclosed herein along a second predetermined path for a time sufficient to create a second etching beginning at least partially at the first predetermined depth for a time period sufficient to create an etching at a second predetermined depth.

### BRIEF DESCRIPTION OF THE DRAWINGS

FIG. 1. The figure shows a representative diagram of a softgel capsule with laser etching on different surfaces and containing etches according to certain embodiments.

FIG. 2. The figure shows representative examples of the method used to measure depth at the etching site.

FIG. 3. The figure shows an example representation of pulsing of the laser combined with modulation of the duty cycles to optimize the laser etching process.

FIG. 4. The figure discloses representative laser angles used to etch softgels according to certain embodiments.

FIG. 5. The figure discloses representative laser angles that can be used to etch softgels according to certain embodiments.

FIGS. 6A and 6B. The figures disclose comparisons of capsule rupture rates between laser-etched capsules and capsules lacking laser etching for 2 different types of softgel capsules (FIG. 6A: Capsule A softgels; FIG. 6B: Capsule B softgels).

FIGS. 7A-7F. The figures disclose images of capsules with and without concentric circles and ovals on their surface.

FIG. 8. The figure discloses an image of a softgel capsule after rupturing along the lines of the channels etched on the surface.

FIGS. 9A and 9B. The figures disclose a mobile, manually fed, laser etching system (FIG. 9A) for processing of softgel capsules and the paddle (FIG. 9B) for holding capsules.

FIGS. 10A-10E. The figures disclose control screens when the system shown in FIG. 9A is in operation. FIG. 10A: Main screen; FIG. 10B: Laser-Image Parameters screen; FIG. 10C: Laser-Laser Parameters screen; FIG. 10D: Laser-Paddle Layout screen; FIG. 10E: Laser-Power Check screen.

FIGS. 11A and 11B. The figures disclose the results of stability testing using blister packs of several embodiments.

FIGS. 12A-12C. The figures disclose the results of disintegration testing. FIG. 12A: Purple softgel disintegration comparison; FIG. 12B: Red capsule (Red-07 and Red-08) disintegration; FIG. 12C: Red capsule (Red-09 and Red-10) disintegration.

### DETAILED DESCRIPTION

In the following description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the scope of the present subject matter. Aspects of the present disclosure, including the Figures, can be arranged, substituted, combined, separated, and designed in a wide variety of different configurations, all of which are contemplated herein.

References in the specification to “one embodiment”, “an embodiment”, “an example embodiment”, or “some embodiments,” etc. indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be effected in connection with other embodiments whether or not explicitly described.

The present disclosure solves problems of prior technologies by providing capsules that provide an increased active ingredient release rate, without exposing the internal chamber of the capsules to the external environment. This makes it possible to enclose APIs that are sensitive to air or oxidation in a capsule that is formulated to immediately deliver API to the GI tract. It also provides manufacturing methods that are readily applicable to capsule formulations already being manufactured or that are in use.

The example compositions and methods described herein overcome one or more of the deficiencies of prior capsule formulations to provide capsules that have on their surface laser-etched furrows that remove a portion of the surface of the capsule, but do not penetrate the capsule and expose the interior contents to the outside environment. These furrows facilitate capsule rupture, disintegration and pharmaceutical release after capsule ingestion without undermining the structural integrity of the capsule during manufacturing, storage, or administration. The disclosed manufacturing methods also optimize ablation of the capsule surface for furrow formation and the generation of structurally sound capsules that have faster drug release profiles.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior invention. Further, the dates of publication provided herein can be different from the actual publication dates, which can require independent confirmation.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, the term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

As used herein, the term, “capsule,” “hard gel capsule,” or “softgel capsule,” refers to the capsule material or capsule shell that is used to encapsulate or enclose the active ingredients, excipients, solvents, and disintegrants contained within. Capsules, whether hard or soft, are made using techniques and materials known in the art. Softgels are commonly used to encapsulate liquids containing active ingredients, such as pharmaceuticals, vitamins, nutrients, or other consumables. Softgels are also used in many other industries, and have been used to encapsulate such diverse substances as industrial adhesives and bath oils.

In the present disclosure, “structural integrity of the capsule” refers to the physical status of the capsule starting immediately after etching is complete, while “stable,” “stability,” and “stability testing,” when referencing softgel capsules, refers to the physical and chemical status of etched softgel capsules as determined using stability testing protocols per CHPA, FDA, or ICH guidelines.

In the present disclosure, “surface of a capsule” or “surface of a softgel” refers to the exterior surface of the capsule or softgel, which surface is in contact with outside environment. “Interior of the capsule” or “interior of the softgel” refers to the inside volume of the capsule or softgel, which is enclosed or enveloped by the inner surface of the capsule shell and is in contact with the ingredients contained inside the capsule shell.

In the present disclosure, “depth” means the distance between the lowest point of the etch (furrow) and the outer shell surface, measured along a normal line perpendicular to a tangent to the outer shell surface at the point of tangency, where the normal line passes through the furrow and the lowest point of the etch. One example of how to measure this can be seen in FIG. 2.

In the present disclosure, “thickness” means the distance between the lowest point of the etch (furrow) and the inner shell surface, measured along a normal line perpendicular to a tangent to the inner shell surface at the point of tangency, where the normal line passes through the furrow and the lowest point of the etch (furrow).

In the present disclosure, “active ingredients” or “API” refers to any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during manufacture and be present in the drug product in a modified form intended to furnish the specified activity or effect and may include for example biologically active substances such as pharmaceuticals, medicaments, minerals, nutraceuticals, vitamins, supplements, amino acids, antioxidants, and similar materials.

The term “rapid release” as applied to tablets or capsules is understood by persons of ordinary skill in the art has structural implications for the respective tablets or capsules. The term is defined, for example, in the current issue of the US Pharmacopoeia (USP), General Chapter 1092, “THE DISSOLUTION PROCEDURE: DEVELOPMENT AND VALIDATION,” heading “STUDY DESIGN,” “Time Points.” For rapid-release or immediate-release dosage forms, the duration of the procedure is typically 30 to 60 minutes; in most cases, a single time point specification is adequate for Pharmacopeia purposes. Industrial and regulatory concepts of product comparability and performance may require additional time points, which may also be required for product registration or approval. A sufficient number of time points should be selected to adequately characterize the ascending and plateau phases of the dissolution curve. According to the Biopharmaceutics Classification System referred to in several FDA Guidances, highly soluble, highly permeable drugs formulated with rapidly dissolving products need not be subjected to a profile comparison if they can be shown to release 85% or more of the active drug substance within 15 minutes. For these types of products, a one-point test will suffice. However, most products do not fall into this category. Dissolution profiles of rapid-release or immediate-release products typically show a gradual increase reaching 85% to 100% at about 30 to 45 minutes. Thus, dissolution time points in the range of 15, 20, 30, 45, and 60 minutes are usual for most rapid-release or immediate-release products.

In some embodiments, under physiological conditions, the capsules or softgels of the disclosure releases at least about 60% of the active ingredient(s) contained therein after about 5 minutes. In some embodiments, under physiological conditions, the capsules or softgels of the disclosure releases at least about 65% of the active ingredient(s) contained therein after about 5 minutes. In some embodiments, under physiological conditions, the capsules or softgels of the disclosure releases at least about 70% of the active ingredient(s) contained therein after about 5 minutes. In some embodiments, under physiological conditions, the capsules or softgels of the disclosure releases at least about 75% of the active ingredient(s) contained therein after about 5 minutes. In some embodiments, under physiological conditions, the capsules or softgels of the disclosure releases at least about 80% of the active ingredient(s) contained therein after about 5 minutes. In some embodiments, under physiological conditions, the capsules or softgels of the disclosure releases at least about 85% of the active ingredient(s) contained therein after about 5 minutes. In some embodiments, under physiological conditions, the capsules or softgels of the disclosure releases at least about 90% of the active ingredient(s) contained therein after about 5 minutes. In some embodiments, under physiological conditions, the capsules or softgels of the disclosure releases at least about 95% of the active ingredient(s) contained therein after about 5 minutes. In some embodiments, under physiological conditions, the capsules or softgels of the disclosure releases more than about 95% of the active ingredient(s) contained therein after about 5 minutes.

In some embodiments, under physiological conditions, the capsules or softgels of the disclosure releases at least about 80% of the active ingredient(s) contained therein after about 5 minutes. In some embodiments, under physiological conditions, the capsules or softgels of the disclosure releases at least about 80% of the active ingredient(s) contained therein after about 10 minutes. In some embodiments, under physiological conditions, the capsules or softgels of the disclosure releases at least about 80% of the active ingredient(s) contained therein after about 15 minutes. In some embodiments, under physiological conditions, the capsules or softgels of the disclosure releases at least about 80% of the active ingredient(s) contained therein after about 20 minutes. In some embodiments, under physiological conditions, the capsules or softgels of the disclosure releases at least about 80% of the active ingredient(s) contained therein after about 25 minutes. In some embodiments, under physiological conditions, the capsules or softgels of the disclosure releases at least about 80% of the active ingredient(s) contained therein after about 30 minutes. In some embodiments, under physiological conditions, the capsules or softgels of the disclosure releases at least about 80% of the active ingredient(s) contained therein after about 35 minutes. In some embodiments, under physiological conditions, the capsules or softgels of the disclosure releases at least about 80% of the active ingredient(s) contained therein after about 40 minutes. In some embodiments, under physiological conditions, the capsules or softgels of the disclosure releases at least about 80% of the active ingredient(s) contained therein after about 45 minutes. In some embodiments, under physiological conditions, the capsules or softgels of the disclosure releases at least about 80% of the active ingredient(s) contained therein after about 50 minutes. In some embodiments, under physiological conditions, the capsules or softgels of the disclosure releases at least about 80% of the active ingredient(s) contained therein after about 55 minutes. In some embodiments, under physiological conditions, the capsules or softgels of the disclosure releases at least about 80% of the active ingredient(s) contained therein after about 60 minutes.

In some embodiments, the release profile, the drug and the pharmaceutical excipients of the capsule or softgel of the disclosure are stable upon storage, such as upon storage at elevated temperature, e.g., 40° C., for 3 months in sealed containers.

In connection with the release profile, “stable” means that when comparing the initial release profile with the release profile after storage, at any given time point the release profiles deviate from one another by not more than about 20%, not more than about 15%, not more than about 10%, not more than about 7.5%, not more than about 5.0%, or not more than about 2.5%.

In connection with the drug and the pharmaceutical excipients, “stable” means that the capsules or softgels satisfy the requirements of EMEA concerning shelf-life of pharmaceutical products.

In some embodiments, the active ingredient may be any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect and may include for example biologically active substances such as pharmaceuticals, medicaments, prodrugs, minerals, nutraceuticals, vitamins, supplements, amino acids, antioxidants, and similar materials.

In some embodiments, the active ingredients may be pharmaceuticals or medicaments including those used in over-the-counter treatments of coughs, colds and other common ailments. Such medicaments are known to persons of skill in the art and may include analgesics or pain relievers, such as NSAIDS (for example, aspirin, acetaminophen, naproxen, and ibuprofen) and “Cox-2” inhibitors (for example, ralfecoxib, rofecoxib, and celecoxib). Other pharmaceuticals or medicaments may include, but are not limited to, cough suppressants, such as dextromethorphan, decongestants, such as pseudoephedrine, and antihistamines, such as chlorpheniramine and doxylamine compounds In some embodiments, the active ingredients may be pharmaceuticals or medicaments that form zwitterions when dissolved with salts that can be used according to the present disclosure are most highly preferred. Other medicaments or supplements that can be used according to the present disclosure include, for example, guaifenesin, loratadine, phenylephrine, lidocaine, clotrimazole, vitamins and minerals, nutritional supplements and other bioactive ingredients.

In addition to the active ingredients, the capsule or softgel of the present disclosure can also contain pharmaceutically acceptable excipients known in the art. A “pharmaceutically acceptable excipient” refers to an ingredient in a pharmaceutical formulation, other than an active ingredient, which is nontoxic to a subject. Pharmaceutically acceptable excipients include any and all solvents, dispersion media, coatings, antibacterial and antifungal agents, isotonic and absorption delaying agents, and the like that are physiologically compatible. Examples of such pharmaceutically acceptable excipients include, but not limited to, the following:
acidifying agents (such as lactic acid, phosphoric acid, acetic acid, citric acid, fumaric acid, hydrochloric acid, malic acid, nitric acid, sulfuric acid, tartaric acid);
alkalizing agents (such as ammonium hydroxide, ammonium carbonate, potassium hydroxide, sodium bicarbonate, sodium borate, sodium carbonate, diethanolamine, sodium hydroxide and trolamine);
antimicrobials (such as benzoic acid, benzyl alcohol, benzalkonium chloride, benzethonium chloride, butylparaben, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium propionate, sorbic acid, sodium benzoate, sodium dehydroacetate, thimerosal and thymol);
antioxidants (such as ascorbic acid, ascorbyl palmitate, butylated hydroxyanisole, butylated hydroxytoluene, hypophosphorous acid, monothioglycerol, propyl gallate, sodium metabisulfite, sodium thiosulfate, sulfur dioxide, sodium formaldehyde sulfoxylate and tocopherols);
buffering agents (such as acetic acid, ammonium carbonate, ammonium phosphate, boric acid, citric acid, potassium meta phosphate, potassium phosphate monobasic, lactic acid, phosphoric acid, potassium citrate, sodium acetate, sodium citrate, sodium lactate solution, dibasic sodium phosphate, and monobasic sodium phosphate);
chelating agents (such as edetate disodium, ethylenediaminetetraacetic acid and edetic acid);
complexing agents (such as ethylenediaminetetraacetic acid (EDTA), edetic acid, gentisic acid ethanolamide and oxyquinoline sulfate);
diluents (such as calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar and confectioner's sugar);
disintegrants (such as alginic acid, microcrystalline cellulose, croscarmellose sodium, crospovidone, polacrilin potassium, sodium starch glycolate, starch, pregelatinized starch);
desiccants (such as calcium chloride, calcium sulfate, and silicon dioxide);
solubilizing agents (such as acacia, cholesterol, glyceryl monostearate, diethanolamine, lanolin alcohols, lecithin, mono- and diglycerides, monoethanolamine, oleic acid, oleyl alcohol, poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, sorbitan monooleate, sorbitan monopalmitate, sorbitan monostearate and stearic acid);
plasticizers (such as castor oil, diacetylated monoglycerides, diethyl phthalate, glycerin, mono- and di-acetylated monoglycerides, polyethylene glycol, propylene glycol, triacetin and triethyl citrate);
polymers (such as cellulose acetate, alkyl celluloses, hydroxyalkyl cellulose, acrylic polymers and copolymers);
sorbents (such as powdered cellulose, charcoal and purified siliceous earth) and carbon dioxide sorbents (such as barium hydroxide lime and soda lime);
stiffening agents (such as hydrogenated castor oil, cetostearyl alcohol, cetyl alcohol, cetyl esters wax, hard fat, paraffin, polyethylene excipient, stearyl alcohol, emulsifying wax, white wax and yellow wax);
suspending and/or viscosity-increasing agents (such as acacia, agar, alginic acid, aluminum monostearate, bentonite, purified bentonite, magma bentonite, carbomer, carboxymethylcellulose calcium, carboxymethylcellulose sodium, carboxymethylcellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth gum and xanthan gum);
lubricants (such as calcium stearate, glyceryl behenate, magnesium stearate, light mineral oil, polyethylene glycol, sodium stearyl fumarate, stearic acid, purified stearic acid, talc, hydrogenated vegetable oil and zinc stearate);
tonicity agents (such as dextrose, glycerin, mannitol, potassium chloride and sodium chloride);
coating agents (such as cellulose acetate, cellulose acetate phthalate, ethylcellulose, gelatin, hydroxypropyl cellulose, hydroxypropyl methylcellulose, hydroxypropyl methylcellulose, methacrylic acid copolymer, methylcellulose, polyethylene glycol, polyvinyl acetate phthalate, phthalate, sodium carboxymethylcellulose, shellac, sucrose, titanium dioxide, carnauba wax and microcrystalline wax); and
colorants (such as caramel, red, yellow, black and ferric oxide).

This is a non-exclusive list and merely represents classes of excipients that may be used in the oral dosage forms as described herein.

Etching:

As employed in the current disclosure, laser etching involves using a laser beam, calibrated to a specific wavelength, to remove a specified amount of material from the exterior surface of a capsule and create a channel or furrow on the surface of the etched capsule. These channels or furrows have several characteristics that distinguish them from the literature. The channels increase the surface area of the capsule exterior exposed to liquid, decrease the capsule thickness in the vicinity of the channels, modify the structural integrity of the capsule at the channel site, and promote water entrapment and eddying at the etching site. These traits result in increased hydrostatic pressure in the vicinity of the channel and accelerate rupture of the capsule and release of the enclosed APIs when compared with capsules lacking such etching. These laser etched channels or furrows also enable faster rupture without undermining the structural integrity of the capsule during manufacturing, storage, or administration.

At least four factors can play a role in the success of the present disclosure. These include controlling the depth of the etching, controlling the lasing angle, controlling the process parameters during etching, and controlling the generation of certain designs or shapes on the surface of the softgel.

In some embodiments, the laser etched channels or furrows are at a depth from about 106 to about 195 microns; in some embodiments, the laser etched channels or furrows are from about 130 to about 195 microns; and in some embodiments, the laser etched channels or furrows are at a depth from about 160 to about 195 microns. In in some embodiments, the depth of the laser etched channels or furrows is from about 234 to about 288 microns; in some embodiments, the depth of the laser etched channels or furrows is from about 250 to about 288 microns; and in some embodiments, the depth of the laser etched channels or furrows is from about 270 to about 288 microns. In some embodiments, the depth of the laser etched channels or furrows is from about 70% to about 86% of the total depth of the softgel capsule; in some embodiments, the depth of the laser etched channels or furrows is from about 75% to about 86% of the total depth of the softgel capsule; and in some embodiments, the depth of the laser etched channels or furrows is from about 80% to about 86% of the total depth of the softgel capsule. In some embodiments, the depth of the laser etched channels or furrows is from about 27% to about 50% of the total depth of the softgel; in some embodiments, the depth of the laser etched channels or furrows is from about 35% to about 50% of the total of the depth of the softgel; and in some embodiments, the depth of the laser etched channels or furrows is from about 40% to about 50% of the total of the depth of the softgel. The laser etching process can also use specific angles and/or channel geometries to optimize the removal of capsule material at the site of the channel or furrow.

In some embodiments, the thickness of the capsule at the etching site is from about 100 to about 46 microns; in some embodiments, the thickness of the capsule at the etching site is from about 85 to about 46 microns; in some embodiments, the thickness of the capsule at the etching site is from about 65 to about 46 microns. In some embodiments, the thickness of the capsule at the etching site is from about 276 to about 187 microns; in some embodiments, the thickness of the capsule at the etching site is from about 240 to about 187 microns; and in some embodiments, the thickness of the capsule at the etching site is from about 210 to about 187 microns. In some embodiments, the thickness of the capsule at the site of the channel or furrow is from about 30% to about 14% of the total capsule thickness; in some embodiments, the thickness of the capsule at the site of the channel or furrow is from about 25% to about 14% of the total capsule thickness; in some embodiments, the thickness of the capsule at the site of the channel or furrow is from 20% to about 14% of the total capsule thickness. In some embodiments, the thickness of the capsule at the site of the channel or furrow is from about 73% to about 50% of the total capsule thickness; in some embodiments, the thickness of the capsule at the site of the channel or furrow is from about 65% to about 50% of the total capsule thickness; and in some embodiments, the thickness of the capsule at the site of the channel or furrow is from about 58% to about 50% of the total thickness of the capsule at the etching site.

The laser etched channels of the present disclosure stand in sharp contrast to laser engraving or marking techniques disclosed in the literature, which used lasers to remove up to about 50 μm of material. See, e.g., U.S. Pub. No. 20130244002 (providing methods of laser marking of capsules for anticounterfeiting that removes between 20-50 μm, or about 5-15%, of the total material from a capsule surface). The laser engraving methods disclosed in the literature act to identify an engraved capsule, but they do not alter the capsule surface in a manner that facilitates capsule rupture or disintegration. Consequently, the laser engraving or marking methods disclosed in the literature also do not facilitate release of the capsule contents. In addition, the literature does not disclose methods or techniques for optimizing the laser ablation of capsule material in a manner necessary to create the furrows or channels of the present disclosure. Regardless, the inventors discovered that an important element in the etching process is controlling the depth of the etch. Controlling the depth prevents the etching process from disrupting the shell or causing leaking of the contents, while still creating channels that modify the structural integrity of the capsule.

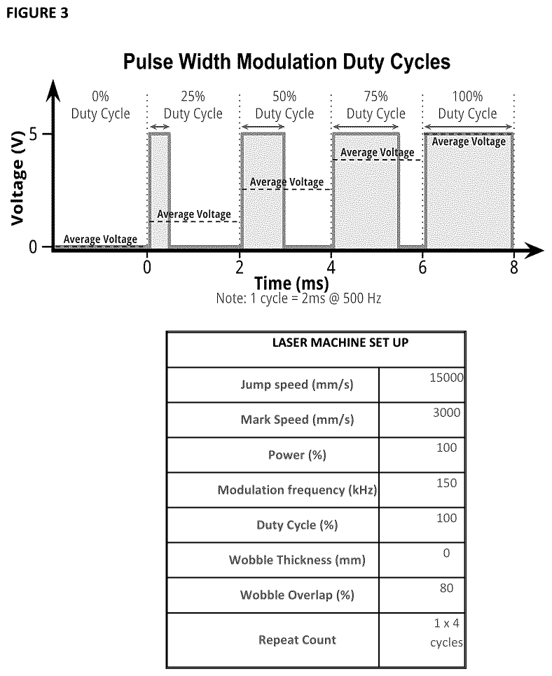

In the present disclosure, the focal or target point for the laser or laser beam is the site on the surface of the capsule where the capsule material is being removed or ablated. Lasing factors can be altered to control the melting process according to methods that would be known to those of ordinary skill based on the disclosure provided herein. For example, a duty cycle illustrated in FIG. 3 shows one such factor that can be altered. In addition, different types of lasers can be used according to the present disclosure. In some embodiments, a CO₂ laser is used, which typically emits at a wavelength of 10.6 μm, but it can vary from about 9 μm to about 11 μm. In some embodiments, the wavelength is about 9.6 μm. The inventors have found that factors inherent in the softgel capsules such as color, thickness, shape, and ingredients can affect the rate of melting of the softgel due to, for example, by varying the rate of softgel removal, the amount of material that needs to be removed, and gel flow during removal. For example, presence of color agents in the gelatin shell, such as transition metals, such as iron, copper, titanium etc., can deflect the laser as sparks from the softgel surface, thereby requiring careful adjusting of laser parameters to generate an effective etching pattern. A person of ordinary skill would understand how to vary the output, power, wavelength, duty cycle, laser distance, angle of incidence, and other lasing control elements according to methods known in the art and in view of the present disclosure.

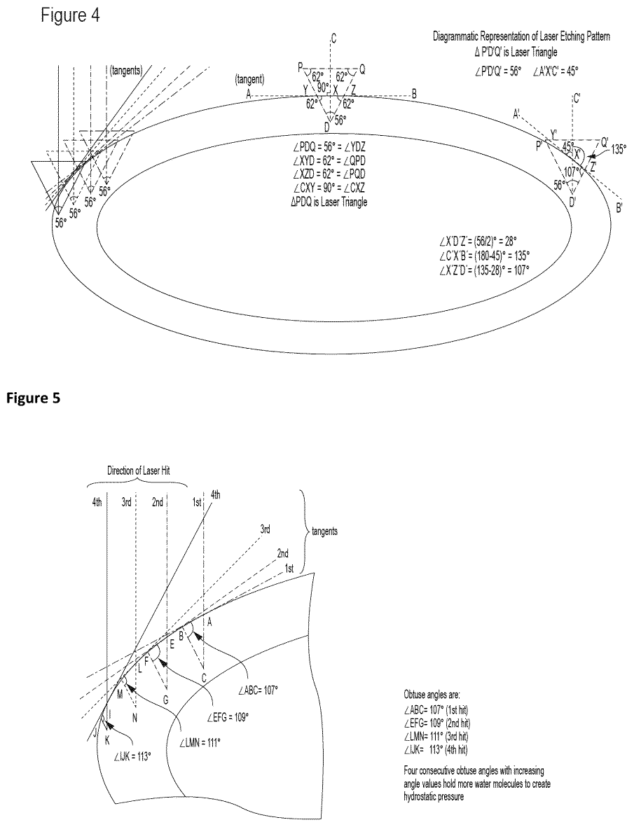

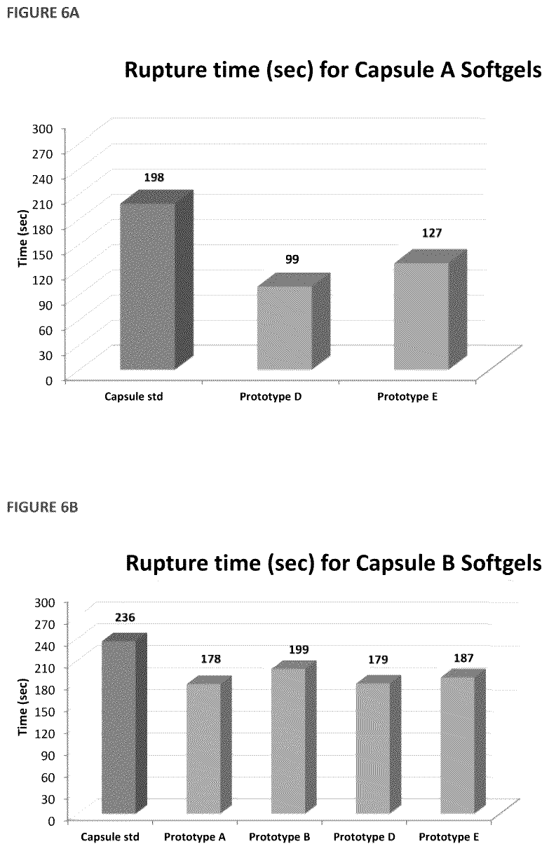

During the ablation process, the laser system can redirect the laser beam to optimize the amount of material removed and the depth of the channels or furrows. One method for optimizing ablation of material during etching is to vary the angle of the laser during etching. The inventors discovered that using multiple passes of the laser at specific, varying angles to generate an etch with an asymmetric angle facilitates rupturing of the softgel capsule and release of the enclosed contents. Representative embodiments illustrating this can be seen in FIG. 4 and FIG. 5. FIG. 5, for example, shows a series of different laser channels that are marked 1^{st} through 4^{th}. Each of these representative embodiments shows a different lasing angle, which varies from about 107° to about 113°.

FIG. 4 discloses different embodiments of the disclosure using different lasing angles. The shape depicts 2 concentric ovoids, which provide a representation of the outer and inner surfaces of a softgel, where the void in the center of the interior ovoid represents the inside of a typical softgel capsule. The line at the center of the apex of the outer ovoid depicts a channel or furrow that can be generated on the upper surface of a softgel according to the present disclosure. The line going from C to D depicts a perpendicular line that can be used to determine the depth of the depicted channel, with the point D showing the point of the channel used to measure the depth. On the right side of the outer ovoid, another embodiment of an etch is depicted. In this instance, the laser is directed at an acute angle to the surface of the softgel during the etching process, due to in part to the specific geometry of the outer edge of the capsule. Finally, the left side of the figure depicts two aspects of the lasing process used in certain embodiments, e.g., how to either: generate multiple concentric etches on a curved softgel surface or generate a single channel in a softgel using multiple passes of the laser.

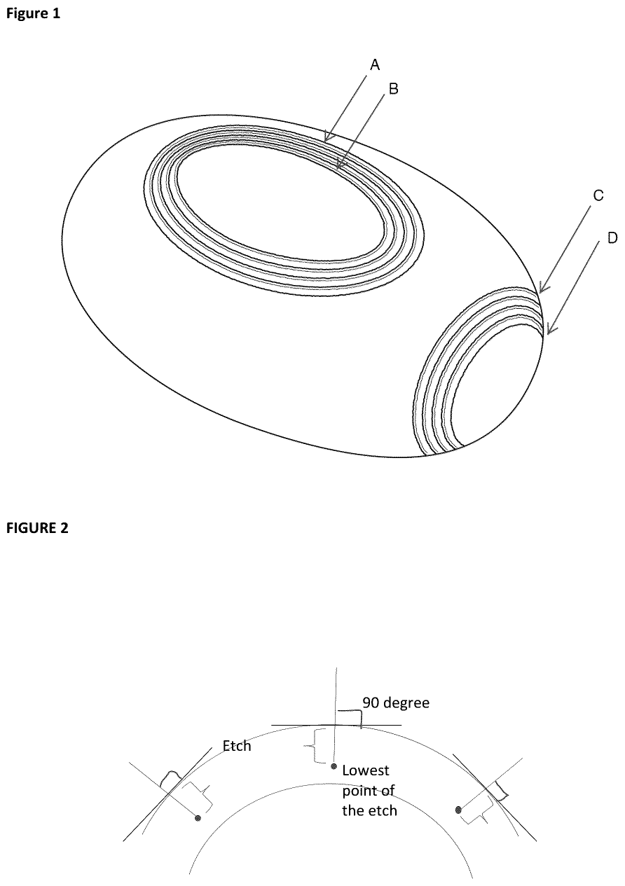

Additional embodiments are also depicted in FIG. 1. In some embodiments, shown in FIG. 1, the laser etched furrows lie on the apical side of the longitudinal axis of the depicted softgel. Also shown in FIG. 1, a representative example of concentric laser etched channels appear in the concentric ovals spanning the channels between A and B. In some embodiments, a single etched channel can be created, which is depicted by the oval lying either at A or B of FIG. 1. In some embodiments, as is provided at C and D of FIG. 1, laser etched channels can be situated at one end of a laser etched capsule. Once again, the capsule can contain a single etched furrow or channel, as depicted by the circle marked C or D of FIG. 1, or it can have multiple concentric etches, as depicted by the concentric circles spanning the space from C to D of FIG. 1. Some embodiments use only a single laser etch on the apical surface of a softgel, and other embodiments use multiple laser etched circles, ovals, or combinations of both on the apical surface of the softgel.

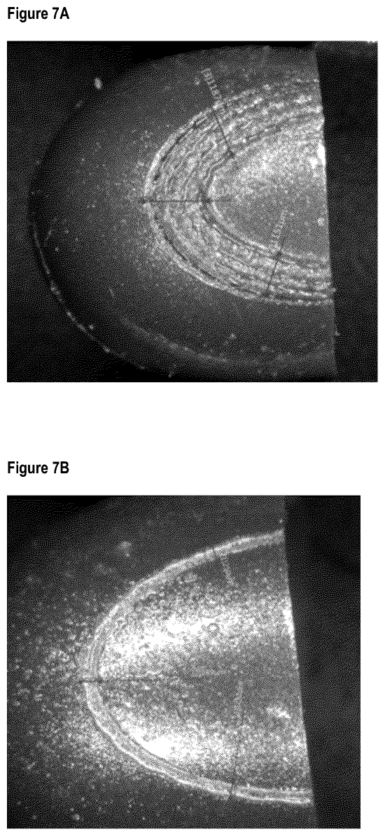

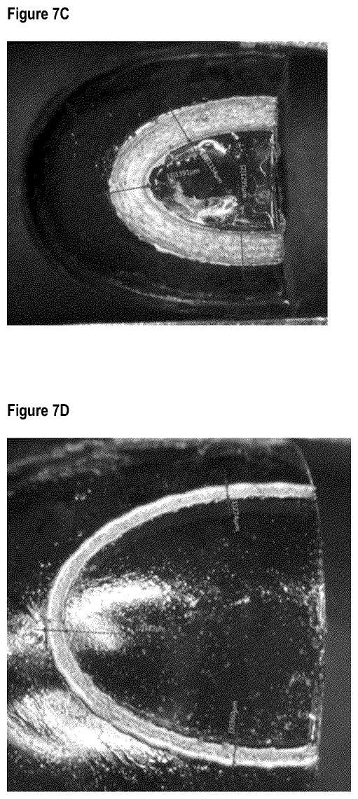

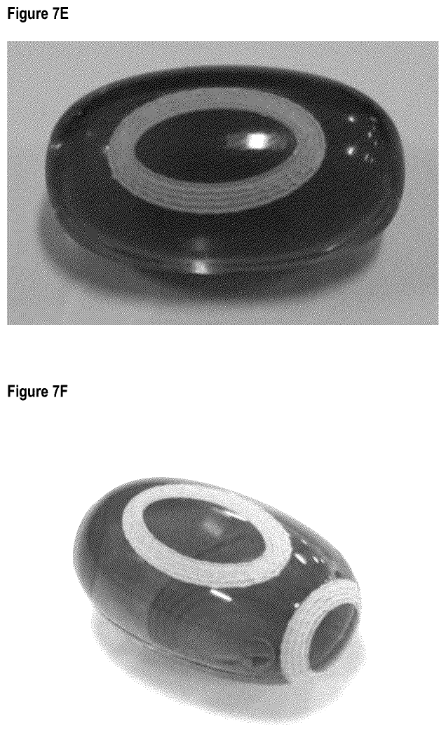

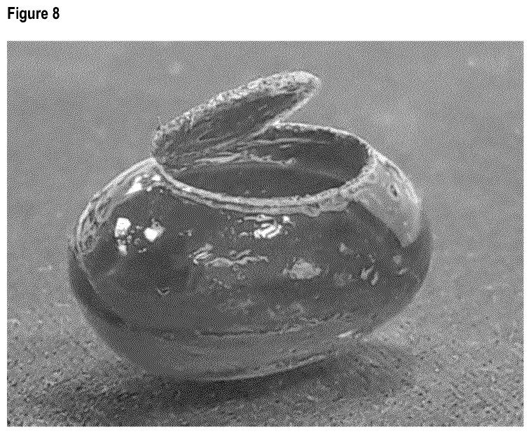

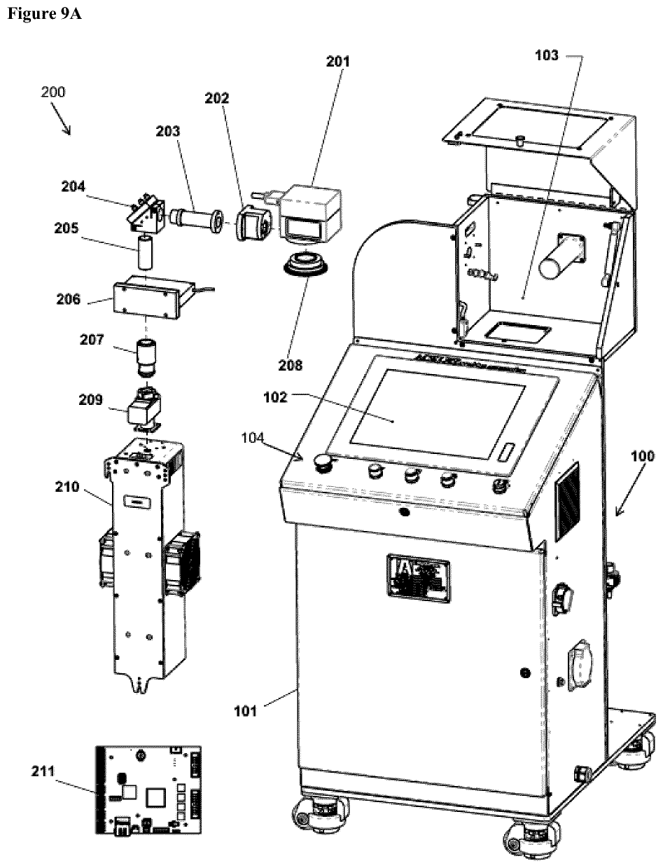

Concerning the depiction of concentric etches, FIG. 5 shows a process for generating concentric etches that progress outward from a point near the edge of a softgel, it is necessary to vary the laser angle with respect to the surface of the softgel. This can be seen in the variation in the angles between the incoming lines (which bisect the triangles) and the tangents lying on the surface at the point of contact. This can be visualized in FIG. 5, which is explained further below. According to some disclosed embodiments, the angle of incidence increases from about 107° to about 113° as the laser progresses from interior to exterior. Other angles and variations may be required according to different capsule geometries and compositions. Images of softgels etched according to this process can be seen in FIGS. 1 and 7.

Concerning the depiction of multiple passes or pulsing of a laser, the principles discussed above also apply to methods for generating a broader channel or furrow situated near to the outer edges of a capsule. The inventors found that it can be advantageous to direct the laser at an angle that is not perpendicular at the point of contact to generate a channel or furrow. The inventors also discovered that multiple passes might be required to achieve the depth and width desired for a single furrow. When applied to the curved surfaces that lie near the outer edges of a capsule, embodiments that forma a single channel using multiple laser passes would also require changing the lasing angle. This is evident from FIGS. 4 and 5.

When multiple passes are used to achieve a desired depth and/or width for a single furrow, the interval between the passes may be from about 0.1 second to about 1 second. In some embodiments, the interval between the passes is from about 0.2 second to about 0.9 second. In some embodiments, the interval between the passes is from about 0.3 second to about 0.8 second. In some embodiments, the interval between the passes is from about 0.4 second to about 0.7 second. In some embodiments, the interval between the passes is from about 0.5 second to about 0.6 second. In some embodiments, the interval between the passes is about 0.1 second. In some embodiments, the interval between the passes is about 0.2 second. In some embodiments, the interval between the passes is about 0.3 second. In some embodiments, the interval between the passes is about 0.4 second. In some embodiments, the interval between the passes is about 0.5 second. In some embodiments, the interval between the passes is about 0.6 second. In some embodiments, the interval between the passes is about 0.7 second. In some embodiments, the interval between the passes is about 0.8 second. In some embodiments, the interval between the passes is about 0.9 second. In some embodiments, the interval between the passes is about 1 second.

The inventors also discovered that etches with asymmetric angles can increase water entrapment by capillary force, and correspondingly increase the hydrostatic force on the etch, which can accelerate rupture of the etched softgel, when compared with etches having symmetric angles. Symmetric angles refer to the angles formed when a laser is positioned perpendicular to a tangent drawn at the surface of the softgel and the shape of the etch at a line drawn through the point of contact is relatively symmetrical. See, for example, FIG. 4, which depicts examples of different types of angles used according to some embodiments. In triangle PQD, for example, a hypothetical laser pulse hits the capsule surface at about 90° to the tangent drawn from line A to B at the point of contact. As shown in the image, a hypothetical laser contacting the gel in this manner would bisect the angle at point D, thereby creating a symmetric angle.

Asymmetric angles refer to the angles formed when the laser is directed at either an obtuse or acute angle to a tangent drawn at the point of contact. Certain embodiments of this can be seen in FIG. 5, which depicts four different asymmetric angles. In the first triangle (with angles ABC), the point of contact of the laser occurs at an angle of about 107°. In the second triangle (with angles EFG), the point of contact of the laser occurs at an angle of 109°. In the third (with angles LMN), the point of contact of the laser occurs at an angle of about 111°. And in the fourth triangle (with angles IJK), the point of contact of the laser occurs at an angle of about 113°. When used in this manner, the laser generates an etch with a relatively asymmetrical shape. The beneficial effects of such embodiments can be seen in Table 1.

TABLE 1
Comparative data for softgels with and without special etching design

| | | | | Capsule B | Capsule B | |
|---|---|---|---|---|---|---|
| | | | | symmetrical | asymmetrical | |
| | | Capsule A | Capsule A | etch (17.5 × 2 = | etch (10 + 25 = | |
| | Capsule A | symmetrical | asymmetrical | 35 degrees) − | 35 degrees) − | Capsule B |
| | control | etch | etch | only one ellipse | only one ellipse | Control |
| Time in seconds | 198 seconds | 195 seconds | 99 seconds | 219 seconds | 178 seconds | 236 seconds |
| to shell rupture | | (prototype B) | (prototype D) | (prototype F) | (prototype A) | |
| (Disintegration | | 185 seconds | 127 seconds | 227 seconds | 179 seconds | Capsule B |
| Time) | | (Prototype C) | (Prototype E) | (prototype H) | (prototype D) | Control |
| Reduction | | | 49% | | 24% | |
| in Time | | | (Average) | | (Average) | |

Notes:
Capsule A is a liquid softgel capsule that is sold commercially as Alka Selzer Pluss; Capsule B is an opaque soft gels (which contains colorants and more gelatin than Capsule A). Statistically significant reduction in disintegration time was noted for ASP softgels for prototypes A & B against ASP control; Statistically significant reduction in disintegration time was noted for PODs softgels for prototypes D & E against PODs control

The system of the present disclosure carefully regulates the path or trace of the laser during ablation to achieve a consistent channel depth throughout the length of the channel and to optimize material removal. The inventors determined that using special designs or shapes on the surface of the capsules can facilitate capsule disintegration. In some embodiments, etching is formed in the shape of concentric ovals or circles on the surface of the softgel. See, for example, FIGS. 1 and 7. FIGS. 7A, 7C, and 7E, for example, disclose capsules with four concentric furrows etched onto the surface in an ovoid shape. FIGS. 7B and 7D disclose a capsule with a single furrow etched onto the surface in an ovoid shape. And FIGS. 1 and 7F discloses a capsule with four concentric furrows etched on four surfaces of the capsule. The inventors discovered that shapes such as these facilitate softgel rupture and API release. Some embodiments of this phenomenon can be seen, for example, in FIG. 8.

Achieving and maintaining stability of etched softgels while they remain in storage is also important. The inventors determined that removal of excessive amounts of softgel material, for example, by using too many concentric etches or using complex surface designs can cause instability and leaking during long term storage. To address this, the inventors found that limiting the number of etches on any particular softgel surface can confer an advantage during long term storage.

In some embodiments, lasing the softgels in the shape of ovoids on the apical surface conferred stability during storage that was similar to softgels lacking etching, while still facilitating rupture and disintegration of the etched softgels. Table 8 for example, shows two different types of softgels with laser etching on the apical surface (similar to what is seen in FIG. 1A), where standard stability tests show that the softgels would remain stable in storage for the equivalent of 2 years or more. The inventors found that the etched softgels of such embodiments still achieved faster rupture and disintegration than similar gel capsules without etching. In addition, softgels having either a single etch on the apical surface (similar to the softgel depicted in FIG. 7B) or two etches on the apical surface, showed faster rupture and disintegration times while maintaining long term stability comparable to unetched softgels when subjected to stability testing; see, e.g., Table 13.

In some embodiments, the laser etched channels are situated on the capsule such that they do not contact or transect any seams in the capsule that were generated during the softgel manufacturing process. Such embodiments are particularly useful for softgel capsules that are in the form of oblate spheroids or of capsules that are otherwise flattened along one plane. Such capsules would appear to have a dorsal and ventral surface depending on the orientation and are elongated in one plane, which often has a seam running along it. During the typical manufacturing process for oblate spheroid capsules, an upper and lower shell are combined and sealed forming a seam along the circle formed by a plane passing through the widest circle of the capsule. In such embodiments, the laser etched furrows would lie on the dorsal or ventral surface of the capsule and would not contact or transect the seam bonding together the 2 halves of the softgel at the line of the plane. Other embodiments include different softgel types or shapes that also contain seams, and in these embodiments the etching does not intersect with the seam formed during manufacturing. For example, some softgels are in the shape of prolate spheroids, spheres, ovals or oblong varieties, which also often contain seams where two halves of the final product were merged together during manufacturing. Certain embodiments include these types of softgels that have laser etched furrows or channels, which do not intersect the softgel seams on those embodiments. In additional embodiments, the laser etched furrows or channels intersect the softgel seams.

In other embodiments, the laser is used to form discontinuous furrows or channels along the path of an etch on the surface of the softgel. The resulting pattern is formed by disrupting the lasing process at intervals to generate a discontinuous line of channels as prescribed by a particular design. Such discontinuous etches are distinguished from prior technologies where holes were drilled through the capsule surface to expose the interior contents. The discontinuous etches of the present disclosure, by contrast, do not penetrate the softgel capsule and, thus, the interior softgel contents are not exposed to the exterior environment.

In some embodiments, the lasing process is disrupted at intervals from about 0.1 second to about 1 second. In some embodiments, the lasing process is disrupted at intervals from about 0.2 second to about 0.9 second. In some embodiments, the lasing process is disrupted at intervals from about 0.3 second to about 0.8 second. In some embodiments, the lasing process is disrupted at intervals from about 0.4 second to about 0.7 second. In some embodiments, the lasing process is disrupted at intervals from about 0.5 second to about 0.6 second. In some embodiments, the lasing process is disrupted at intervals of about 0.1 second. In some embodiments, the lasing process is disrupted at intervals of about 0.2 second. In some embodiments, the lasing process is disrupted at intervals of about 0.3 second. In some embodiments, the lasing process is disrupted at intervals of about 0.4 second. In some embodiments, the lasing process is disrupted at intervals of about 0.5 second. In some embodiments, the lasing process is disrupted at intervals of about 0.6 second. In some embodiments, the lasing process is disrupted at intervals of about 0.7 second. In some embodiments, the lasing process is disrupted at intervals of about 0.8 second. In some embodiments, the lasing process is disrupted at intervals of about 0.9 second. In some embodiments, the lasing process is disrupted at intervals of about 1 second.

In some embodiments, a laser controller directs a laser beam of a specific wavelength at capsules that are situated on a surface. The controller and the surface can rotate and move in a manner that controls the angle, depth, and length of the channels or furrows created when the laser ablates material from the surface of the capsule. In some embodiments, capsules are mounted on workpieces, which can be cylindrical or can be flat workpieces or any other suitable capsule holder with or without perforations, and the controller directs a laser beam to create a specified design and achieve the desired channels or furrows. Such a set up can be also be upgraded so that two lasers can fire targeting both the surfaces (upper and lower) simultaneously, thereby creating two similar or different designs in each side of the capsule in the same run time. In some embodiments, both the laser and the surface are stationary, and galvo mirrors are employed to direct the laser beam over the capsule surface and achieve the desired channel depth. Other embodiments that vary the orientation of the laser, placement of a softgel on a surface, or mounting on a workpiece would be known to persons of ordinary skill in the art based on the present disclosure. In addition, the system of the present disclosure can remove ablated material from the vicinity of the ablation site through a variety of methods, such as, by using ventilation systems, vacuums, or blowers.

In any of the above embodiments, the controller can alter the timing and pulsing of the laser during the ablation process, as required by the specific apparatus and capsule materials. The system of the present disclosure controls tightly the speed, power, and timing of the laser pulses across the surface of the capsule to achieve the depth and angles required by certain embodiments. The inventors determined that pulsing of the laser at certain wavelengths and power settings can facilitate the creation of surface channels. At certain laser settings, lasing with a constant beam would melt the softgel material beyond the immediate vicinity of the etch. The resulting melted material would then flow into the channels or furrows and re-cast the outer layer of the softgel in a manner that could reduce the effectiveness of the etching process.

The inventors determined that lasing with short bursts or pulses at high intensity settings could generate etching with the desired characteristics. By pulsing the laser and modulating the length of the pulse or cycle at certain settings, the so-called “duty cycle,” the inventors determined that they could eliminate recasting of melted softgel material. An illustration of a duty cycle can be seen in FIG. 3, which discloses a series of laser pulses represented by the square waves of increasing duration of output (beginning at zero and increasing to 100%). By modulating the amount of time that a laser is “on”, the inventors were able to alter the duty cycle and generate channels with the desired characteristics while reducing the amount of flow of melted softgel that flows back into the nascent channel.

In some embodiments, the laser beam is directed towards a rotating or vibrating mirror. The mirror moves in a manner which may trace out patterns onto the surface being etched and enables the marking of materials that are not stationary. Another embodiment uses a stationary surface during capsule etching. The laser can be stationary and emit light toward movable mirrors. The mirrors can be situated on a movable surface and can have variable angles of reflection of the laser beam. These variables can be altered in a manner that make it possible to pass the laser over every point of the stationary surface.

Some other embodiments can employ dynamic auto focus systems, which can alter the lasing parameters (such as height, depth, intensity, etc.) during the lasing process to optimize channel characteristics. The specific embodiments provided here are non-limiting illustrative examples.

Some other embodiment discloses a capsule comprising: at least one laser etched furrow situated on the surface of the capsule, wherein the furrow penetrates at least partially into the surface of the capsule while maintaining the structural integrity of the capsule and without passing through to the interior of the capsule, and wherein the furrow facilitates capsule rupture and disintegration and increases the rate of release of the active ingredients contained within.

In some embodiments, the disclosure discloses a capsule comprising: more than one laser etched furrow situated on the surface of the capsule, wherein the furrows penetrate at least partially into the surface of the capsule while maintaining the structural integrity of the capsule and without passing through to the interior of the capsule, and wherein the furrows facilitate capsule rupture and disintegration and increase the rate of release of the active ingredients contained within.

In some embodiments, the disclosure discloses a capsule comprising: at least one laser etched furrow situated on the surface of the capsule, wherein the at least one furrow penetrates at least partially into the surface of the capsule while maintaining the structural integrity of the capsule and preventing exposure of the interior contents to the outside environment, and wherein the at least one furrow facilitates capsule rupture and disintegration, and increases the rate of release of the active ingredients contained within.

In some embodiments, the disclosure discloses a capsule comprising: at least one laser etched furrow situated on the surface of the capsule, wherein the at least one furrow penetrates at least partially into the surface of the capsule while maintaining the structural integrity of the capsule and without passing through to the interior of the capsule, and wherein the at least one furrow facilitates capsule rupture and disintegration by increasing the surface area of the capsule exposed to solvent (water), entrapping water molecules to create hydrostatic pressure, increasing rate of disintegration of capsule material at the site of the furrow, and promoting capsule erosion at the etching site.

In some embodiments, the disclosure discloses a capsule comprising: at least one laser etched furrow situated on the surface of the capsule, wherein the at least one furrow penetrates at least partially into the surface of the capsule while maintaining the structural integrity of the capsule and preventing exposure of the interior contents to the outside environment, and wherein the at least one furrow facilitates the capsule disintegration rate by increasing the surface area of the capsule exposed to solvent, and promoting capsule erosion at the etching site.

In some embodiments, the disclosure discloses a capsule comprising: at least one laser etched furrow situated on the surface of the capsule, wherein the at least one furrow penetrates at least partially into the surface of the capsule while maintaining the structural integrity of the capsule and preventing exposure of the interior contents to the outside environment and wherein the at least one furrow facilitates active ingredient release during rupture and disintegration of the capsule.

In some embodiments, the disclosure discloses a capsule comprising: at least one laser etched furrow situated on the surface of the capsule, wherein the at least one furrow penetrates at least partially into the surface of the capsule while maintaining the structural integrity of the capsule and preventing exposure of the interior contents to the outside environment, wherein the at least one furrow facilitates active ingredient release during disintegration of the capsule, and wherein the at least one furrow was generated using at least one laser positioned during ablation at an angle that is greater than 90° to a tangent drawn at the point of contact of the laser on the capsule surface.

The disclosure relates to a device comprising a laser, a processor in operable communication with the laser, a paddle for positioning a capsule, and a controller, optionally comprising a computer program product disclosed herein. The laser of the system can ablate capsules positioned at a predetermined distance from the laser at certain wavelengths. In some embodiments, the laser wavelength is from about 9 μm to about 11 μm. In some embodiments, the laser wavelength is from 9.5 μm to about 10.5 μm. In some embodiments, the laser wavelength is about 9 μm. In some embodiments, the laser wavelength is about 9.5 μm. In some embodiments, the laser wavelength is about 10 μm. In some embodiments, the laser wavelength is about 10.5 μm. In some embodiments, the laser wavelength is about 10.6 μm. In some embodiments, the laser wavelength is about 11 μm. In some embodiments, the disclosure relates to a method of making a capsule disclosed herein comprising ablating the capsule surface with a laser for a first predetermined period of time, pausing the ablating for about half a second or more, and then ablating the capsule surface again. The last two steps may be repeated for any number of time necessary to create an etching of one or a plurality of furrows on the capsule of a width and/or depth sufficient to accelerate disintegration of the capsule as compared to a capsule free of the etching.

In some embodiments, the laser power is from about 60 watts to about 100 watts. In some embodiments, the laser power is from about 65 watts to about 95 watts. In some embodiments, the laser power is from about 70 watts to about 90 watts. In some embodiments, the laser power is from about 75 watts to about 85 watts. In some embodiments, the laser power is from about 70 watts to about 80 watts. In some embodiments, the laser power is about 60 watts. In some embodiments, the laser power is about 65 watts. In some embodiments, the laser power is about 70 watts. In some embodiments, the laser power is about 75 watts. In some embodiments, the laser power is about 80 watts. In some embodiments, the laser power is about 85 watts. In some embodiments, the laser power is about 90 watts. In some embodiments, the laser power is about 95 watts. In some embodiments, the laser power is about 100 watts.

In some embodiments, the focused beam spot size range is from about 100 to about 600 micron diameter. In some embodiments, the focused beam spot size range is from about 150 to about 550 micron diameter. In some embodiments, the focused beam spot size range is from about 200 to about 500 micron diameter. In some embodiments, the focused beam spot size range is from about 250 to about 450 micron diameter. In some embodiments, the focused beam spot size range is from about 300 to about 400 micron diameter. In some embodiments, the focused beam spot size range is about 100 micron diameter. In some embodiments, the focused beam spot size range is about 150 micron diameter. In some embodiments, the focused beam spot size range is about 200 micron diameter. In some embodiments, the focused beam spot size range is about 250 micron diameter. In some embodiments, the focused beam spot size range is about 300 micron diameter. In some embodiments, the focused beam spot size range is about 350 micron diameter. In some embodiments, the focused beam spot size range is about 400 micron diameter. In some embodiments, the focused beam spot size range is about 450 micron diameter. In some embodiments, the focused beam spot size range is about 500 micron diameter. In some embodiments, the focused beam spot size range is about 550 micron diameter. In some embodiments, the focused beam spot size range is about 600 micron diameter.

In some embodiments, the power level (duty cycle) setting may range from about 10% to about 90%. In some embodiments, the power level (duty cycle) setting may range from about 15% to about 85%. In some embodiments, the power level (duty cycle) setting may range from about 20% to about 80%. In some embodiments, the power level (duty cycle) setting may range from about 25% to about 75%. In some embodiments, the power level (duty cycle) setting may range from about 30% to about 70%. In some embodiments, the power level (duty cycle) setting may range from about 35% to about 65%. In some embodiments, the power level (duty cycle) setting may range from about 40% to about 60%. In some embodiments, the power level (duty cycle) setting may range from about 45% to about 55%. In some embodiments, the power level (duty cycle) setting is about 10%. In some embodiments, the power level (duty cycle) setting is about 15%. In some embodiments, the power level (duty cycle) setting is about 20%. In some embodiments, the power level (duty cycle) setting is about 25%. In some embodiments, the power level (duty cycle) setting is about 30%. In some embodiments, the power level (duty cycle) setting is about 35%. In some embodiments, the power level (duty cycle) setting is about 40%. In some embodiments, the power level (duty cycle) setting is about 45%. In some embodiments, the power level (duty cycle) setting is about 50%. In some embodiments, the power level (duty cycle) setting is about 55%. In some embodiments, the power level (duty cycle) setting is about 60%. In some embodiments, the power level (duty cycle) setting is about 65%. In some embodiments, the power level (duty cycle) setting is about 70%. In some embodiments, the power level (duty cycle) setting is about 75%. In some embodiments, the power level (duty cycle) setting is about 80%. In some embodiments, the power level (duty cycle) setting is about 85%. In some embodiments, the power level (duty cycle) setting is about 90%.

In some embodiments, the pulse frequency setting is from about 10 kHz to about 100 kHz. In some embodiments, the pulse frequency setting is from about 15 kHz to about 95 kHz. In some embodiments, the pulse frequency setting is from about 20 kHz to about 90 kHz. In some embodiments, the pulse frequency setting is from about 25 kHz to about 85 kHz. In some embodiments, the pulse frequency setting is from about 30 kHz to about 80 kHz. In some embodiments, the pulse frequency setting is from about 35 kHz to about 75 kHz. In some embodiments, the pulse frequency setting is from about 40 kHz to about 70 kHz. In some embodiments, the pulse frequency setting is from about 45 kHz to about 65 kHz. In some embodiments, the pulse frequency setting is from about 50 kHz to about 60 kHz. In some embodiments, the pulse frequency setting is about 10 kHz. In some embodiments, the pulse frequency setting is about 15 kHz. In some embodiments, the pulse frequency setting is about 20 kHz. In some embodiments, the pulse frequency setting is about 25 kHz. In some embodiments, the pulse frequency setting is about 30 kHz. In some embodiments, the pulse frequency setting is about 35 kHz. In some embodiments, the pulse frequency setting is about 40 kHz. In some embodiments, the pulse frequency setting is about 45 kHz. In some embodiments, the pulse frequency setting is about 50 kHz. In some embodiments, the pulse frequency setting is about 55 kHz. In some embodiments, the pulse frequency setting is about 60 kHz. In some embodiments, the pulse frequency setting is about 65 kHz. In some embodiments, the pulse frequency setting is about 70 kHz. In some embodiments, the pulse frequency setting is about 75 kHz. In some embodiments, the pulse frequency setting is about 80 kHz. In some embodiments, the pulse frequency setting is about 85 kHz. In some embodiments, the pulse frequency setting is about 90 kHz. In some embodiments, the pulse frequency setting is about 95 kHz. In some embodiments, the pulse frequency setting is about 100 kHz.

In some embodiments, the scanner marking speed setting is from about 1,000 mm/s to about 8,000 mm/s. In some embodiments, the scanner marking speed setting is from about 1,500 mm/s to about 7,500 mm/s. In some embodiments, the scanner marking speed setting is from about 2,000 mm/s to about 7,000 mm/s. In some embodiments, the scanner marking speed setting is from about 2,500 mm/s to about 6,500 mm/s. In some embodiments, the scanner marking speed setting is from about 3,000 mm/s to about 6,000 mm/s. In some embodiments, the scanner marking speed setting is from about 3,500 mm/s to about 5,500 mm/s. In some embodiments, the scanner marking speed setting is from about 4,000 mm/s to about 5,000 mm/s. In some embodiments, the scanner marking speed setting is about 1,000 mm/s. In some embodiments, the scanner marking speed setting is about 1,500 mm/s. In some embodiments, the scanner marking speed setting is about 2,000 mm/s. In some embodiments, the scanner marking speed setting is about 2,500 mm/s. In some embodiments, the scanner marking speed setting is about 3,000 mm/s. In some embodiments, the scanner marking speed setting is about 3,500 mm/s. In some embodiments, the scanner marking speed setting is about 4,000 mm/s. In some embodiments, the scanner marking speed setting is about 4,500 mm/s. In some embodiments, the scanner marking speed setting is about 5,000 mm/s. In some embodiments, the scanner marking speed setting is about 5,500 mm/s. In some embodiments, the scanner marking speed setting is about 6,000 mm/s. In some embodiments, the scanner marking speed setting is about 6,500 mm/s. In some embodiments, the scanner marking speed setting is about 7,000 mm/s. In some embodiments, the scanner marking speed setting is about 7,500 mm/s. In some embodiments, the scanner marking speed setting is about 8,000 mm/s.

In some embodiments, the repeat count setting is from about 1 to about 10. In some embodiments, the repeat count setting is from about 2 to about 9. In some embodiments, the repeat count setting is from about 3 to about 8. In some embodiments, the repeat count setting is from about 4 to about 7. In some embodiments, the repeat count setting is from about 5 to about 6. In some embodiments, the repeat count setting is about 1. In some embodiments, the repeat count setting is about 2. In some embodiments, the repeat count setting is about 3. In some embodiments, the repeat count setting is about 4. In some embodiments, the repeat count setting is about 5. In some embodiments, the repeat count setting is about 6. In some embodiments, the repeat count setting is about 7. In some embodiments, the repeat count setting is about 8. In some embodiments, the repeat count setting is about 9. In some embodiments, the repeat count setting is about 10.

In some embodiments, the repeat delay setting is from about 0 to about 700 milliseconds. In some embodiments, the repeat delay setting is from about 50 to about 650 milliseconds. In some embodiments, the repeat delay setting is from about 100 to about 600 milliseconds. In some embodiments, the repeat delay setting is from about 150 to about 550 milliseconds. In some embodiments, the repeat delay setting is from about 200 to about 500 milliseconds. In some embodiments, the repeat delay setting is from about 250 to about 450 milliseconds. In some embodiments, the repeat delay setting is from about 300 to about 400 milliseconds. In some embodiments, the repeat delay setting is about 0 millisecond. In some embodiments, the repeat delay setting is from about 50 milliseconds. In some embodiments, the repeat delay setting is from about 100 milliseconds. In some embodiments, the repeat delay setting is from about 150 milliseconds. In some embodiments, the repeat delay setting is from about 200 milliseconds. In some embodiments, the repeat delay setting is from about 250 milliseconds. In some embodiments, the repeat delay setting is from about 300 milliseconds. In some embodiments, the repeat delay setting is from about 350 milliseconds. In some embodiments, the repeat delay setting is from about 400 milliseconds. In some embodiments, the repeat delay setting is from about 450 milliseconds. In some embodiments, the repeat delay setting is from about 500 milliseconds. In some embodiments, the repeat delay setting is from about 550 milliseconds. In some embodiments, the repeat delay setting is from about 600 milliseconds. In some embodiments, the repeat delay setting is from about 650 milliseconds. In some embodiments, the repeat delay setting is from about 700 milliseconds.

The above-described methods and systems for etching capsules or softgels of the disclosure can be implemented in any of numerous ways. For example, the embodiments may be implemented using a non-transitory computer program product (e.g., software), hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a personal digital assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

A computer employed to implement at least a portion of the functionality described herein may include a memory, coupled to one or more processing units (also referred to herein simply as “processors”), one or more communication interfaces, one or more display units, and one or more user input devices. The memory may include any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow the user to make manual adjustments, make selections, enter data or various other information, and/or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention disclosed herein. The computer readable medium or media can be transportable, such that the program or programs stored thereon can be loaded onto one or more different computers or other processors to implement various aspects of the present invention as discussed above. In some embodiments, the system comprises cloud-based software that executes one or all of the steps of each disclosed method instruction.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

In some embodiments, the disclosure relates to a computer program product encoded on a computer-readable storage medium comprising instructions for executing any of the disclosed methods of etching capsules or softgels as described above. In some embodiments, the disclosure relates to a system that comprises the disclosed computer program product, at least one processor, a program storage, such as memory, for storing program code executable on the processor, and one or more input/output devices and/or interfaces, such as data communication and/or peripheral devices and/or interfaces. In some embodiments, the user device and computer system or systems are communicably connected by a data communication network, such as a local area network (LAN), the Internet, or the like, which may also be connected to a number of other client and/or server computer systems. The user device and client and/or server computer systems may further include appropriate operating system software.

In some embodiments, components and/or units of the devices described herein may be able to interact through one or more communication channels or mediums or links, for example, a shared access medium, a global communication network, the Internet, the World Wide Web, a wired network, a wireless network, a combination of one or more wired networks and/or one or more wireless networks, one or more communication networks, an a-synchronic or asynchronous wireless network, a synchronic wireless network, a managed wireless network, a non-managed wireless network, a burstable wireless network, a non-burstable wireless network, a scheduled wireless network, a non-scheduled wireless network, or the like.

Some embodiments may take the form of a non-transitory computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device.

In some embodiments, the medium may be or may include an electronic, magnetic, optical, electromagnetic, InfraRed (IR), or semiconductor system (or apparatus or device) or a propagation medium. Some demonstrative examples of a computer-readable medium may include a semiconductor or solid state memory, magnetic tape, a removable computer diskette, a random access memory (RAM), a read-only memory (ROM), a rigid magnetic disk, an optical disk, or the like. Some demonstrative examples of optical disks include compact disk read-only memory (CD-ROM), compact disk read/write (CD-R/W), DVD, or the like.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Some embodiments may be implemented, for example, using a machine-readable medium or article which may store an instruction or a set of instructions that, if executed by a machine, cause the machine to perform a method and/or operations described herein. Such machine may include, for example, any suitable processing platform, computing platform, computing device, processing device, electronic device, electronic system, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, compact disk read-only memory (CD-ROM), compact disk recordable (CD-R), compact disk re-writeable (CD-RW), optical disk, magnetic media, various types of digital versatile disks (DVDs), a tape, a cassette, or the like. The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, e.g., C, C++, Java′, BASIC, Pascal, Fortran, Cobol, assembly language, machine code, or the like.

In some embodiments, the disclosure relates to a non-transitory computer program product encoded on a computer-readable storage medium that comprises instructions for performing any of the methods described herein. In some embodiments, the disclosure relates to instructions of: lasing a capsule disclosed herein along a first predetermined path for a time period sufficient to create an etching at a first predetermined depth; pausing the lasing from about 0.1 to about 1 second; lasing the capsule disclosed herein along a second predetermined path for a time sufficient to create a second etching at the first predetermined depth for a time period sufficient to create an etching at a second predetermined depth.

In some embodiments, the disclosure relates to a computer-implemented method of directing the position and activation of a laser, the method comprising: lasing a capsule disclosed herein along a first predetermined path for a time period sufficient to create an etching at a first predetermined depth; pausing the lasing from about 0.1 to about 1 second; lasing the capsule disclosed herein along a second predetermined path for a time sufficient to create a second etching at the first predetermined depth for a time period sufficient to create an etching at a second predetermined depth.

In some embodiments, the instructions of the disclosed non-transitory computer program product or the instructions of the disclosed computer-implemented method repeat the step of pausing the lasing and the step of lasing the capsule until the etching is at a depth of from about 106 to about 195 microns. In some embodiments, the instructions repeat the step of pausing the lasing and the step of lasing the capsule until the etching is at a depth of from about 130 to about 195 microns. In some embodiments, the instructions repeat the step of pausing the lasing and the step of lasing the capsule until the etching is at a depth of from about 160 to about 195 microns. In some embodiments, the instructions repeat the step of pausing the lasing and the step of lasing the capsule until the etching is at a depth of from about 234 to about 288 microns. In some embodiments, the instructions repeat the step of pausing the lasing and the step of lasing the capsule until the etching is at a depth of from about 250 to about 288 microns. In some embodiments, the instructions repeat the step of pausing the lasing and the step of lasing the capsule until the etching is at a depth of from about 270 to about 288 microns. In some embodiments, the instructions repeat the step of pausing the lasing and the step of lasing the capsule for from about 1 to about 5 times.

In some embodiments, the instructions of the disclosed non-transitory computer program product or the instructions of the disclosed computer-implemented method further comprise one or more steps of positioning the laser or a capsule. In some embodiments, the positioning of the laser comprises positioning the laser at one or a plurality of positions capable of ablating a softgel capsule surface to create a line or furrow at one or plurality of predetermined depths at one or a plurality of predetermined angles.

In some embodiments, the instructions of the disclosed non-transitory computer program product or the instructions of the disclosed computer-implemented method further comprise one or more steps of modifying etch type. In some embodiments, the etch type is concentric ellipses. In some embodiments, the etch type is single ellipse around single circle.

In some embodiments, the instructions of the disclosed non-transitory computer program product or the instructions of the disclosed computer-implemented method further comprise one or more steps of modifying the duty cycle of the lasering. In some embodiments, the duty cycle of the lasering is about 100%. In some embodiments, the duty cycle of the lasering is about 75%. In some embodiments, the duty cycle of the lasering is about 50%.

In some embodiments, the instructions of the disclosed non-transitory computer program product or the instructions of the disclosed computer-implemented method further comprise one or more steps of modifying the mark speed of the lasering. In some embodiments, the mark speed is about 2000 mm/s. In some embodiments, the mark speed is about 1500 mm/s. In some embodiments, the mark speed is about 1000 mm/s. In some embodiments, the mark speed is about 100%. In some embodiments, the mark speed is about 95%. In some embodiments, the mark speed is about 90%. In some embodiments, the mark speed is about 85%. In some embodiments, the mark speed is about 80%.

In some embodiments, the instructions of the disclosed non-transitory computer program product or the instructions of the disclosed computer-implemented method further comprise one or more steps of modifying the laser power. In some embodiments, the laser power is about 100%. In some embodiments, the laser power is about 95%. In some embodiments, the laser power is about 90%. In some embodiments, the laser power is about 85%. In some embodiments, the laser power is about 80%. In some embodiments, the laser power is about 75%. In some embodiments, the laser power is about 70%. In some embodiments, the laser power is about 65%. In some embodiments, the laser power is about 60%. In some embodiments, the laser power is about 55%. In some embodiments, the laser power is about 50%.

In some embodiments, the instructions of the disclosed non-transitory computer program product or the instructions of the disclosed computer-implemented method further comprise one or more steps of modifying depth count. In some embodiments, the depth count is about 1. In some embodiments, the depth count is about 2. In some embodiments, the depth count is about 3. In some embodiments, the depth count is about 4. In some embodiments, the depth count is about 5.

In some embodiments, the instructions of the disclosed non-transitory computer program product or the instructions of the disclosed computer-implemented method further comprise one or more steps of modifying the repeat delay of the lasering. In some embodiments, the repeat delay is about 0.1 second. In some embodiments, the repeat delay is about 0.2 second. In some embodiments, the repeat delay is about 0.3 second. In some embodiments, the repeat delay is about 0.4 second. In some embodiments, the repeat delay is about 0.5 second. In some embodiments, the repeat delay is about 0.6 second. In some embodiments, the repeat delay is about 0.7 second. In some embodiments, the repeat delay is about 0.8 second. In some embodiments, the repeat delay is about 0.9 second. In some embodiments, the repeat delay is about 1 second.

In some embodiments, the disclosure provides a system comprising: (a) any of the computer program product disclosed herein, and (b) a processor operable to execute programs; and/or a memory associated with the processor.

In some embodiments, the disclosure provides a device comprising: (a) a laser, and (b) any of the computer program product or system disclosed herein.

### Examples

The below examples provide specific embodiments. The specific embodiments show exemplary capsules that can be made according to the teachings contained herein, but the use of these specific examples is not intended to be limiting.

### Example 1

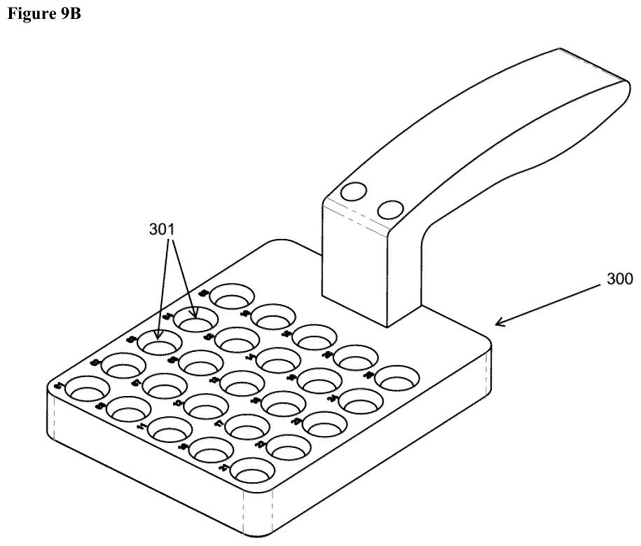

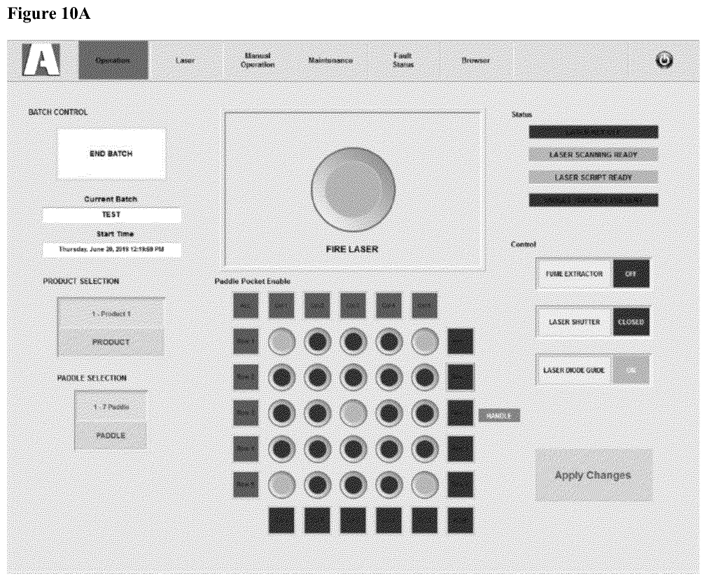

Laser etching of the surface of capsules can be performed using the system described in this example, which is a mobile, manually fed, laser etching system for processing of gelatin type capsules, also referred to as softgels. Referring to FIG. 9A now, the system 100 comprises a base cabinet 101, a control panel 102, a laser processing chamber 103, and a laser assembly 200 comprising a galvanometer scanner 201, a galvanometer scanner mount 202, a beam protection tube 203, a beam bender (mirror) 204, a beam tube 205, a beam safety shutter 206, a beam expander 207, a f-theta scanner objective lens 208, a diode pointer 209, a laser 210, and a laser control board 211.

The system operates by placing tablets (not shown) in a custom made holding device, referred to as a paddle 300 (FIG. 9B). The paddle 300 has a pre-determined amount of pockets 301 to hold the product. The filled paddle is placed into the processing area, referred to as the laser chamber 103. Once inside the laser chamber 103, the safety door to the laser chamber is closed and processing is initiated with a button press on the human machine interface 104, referred to as the HMI.

The system will utilize a pre-determined pattern to ablate into the surface of the softgel, pause for a specified amount of time, then repeat the process based on the repetitions chosen on the HMI. Both the pause time and repetitions are variables than can be set on the HMI. Once the laser processing is complete, the safety enclosure for the laser chamber is opened and the paddle with product is removed.

For the system 100 to function, the setting may include laser parameters and laser settings. In some embodiments, the laser parameters may include laser wavelength, laser power and focused beam spot size range. In some embodiments, the laser settings may include power level (duty cycle), pulse frequency, scanner marking speed, repeat count, and repeat delay.

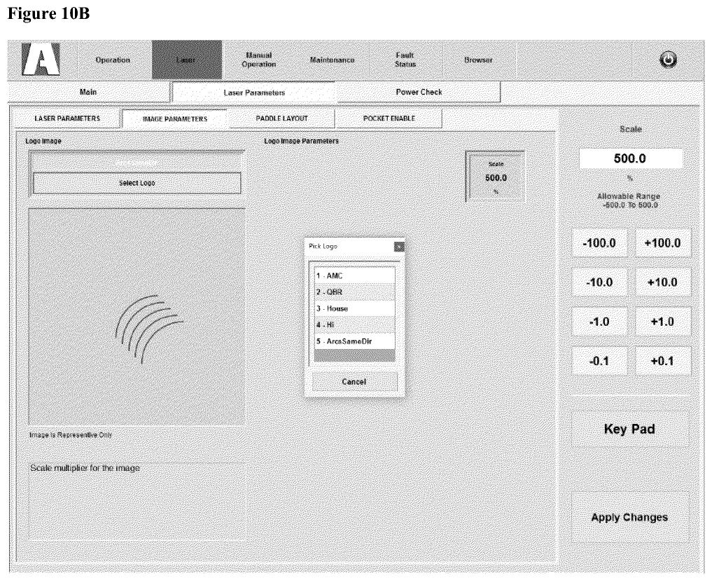

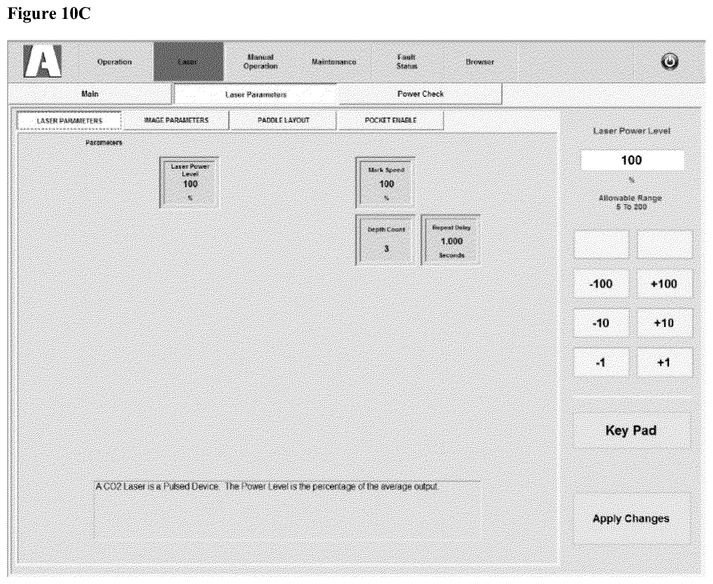

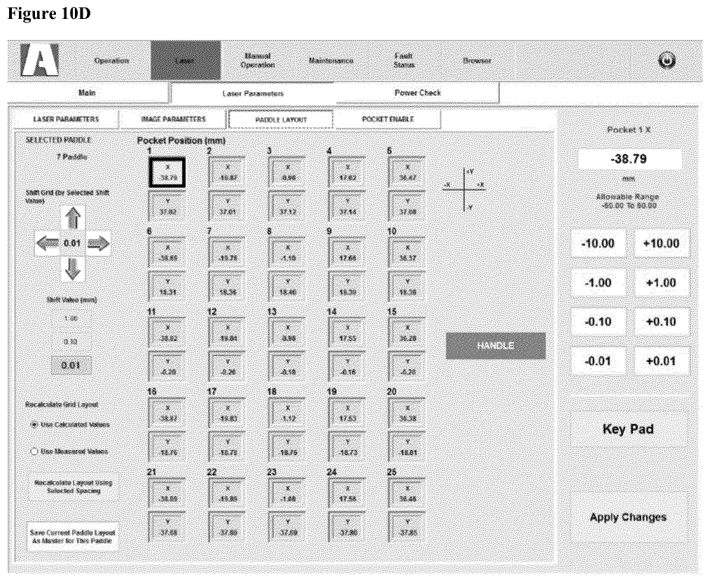

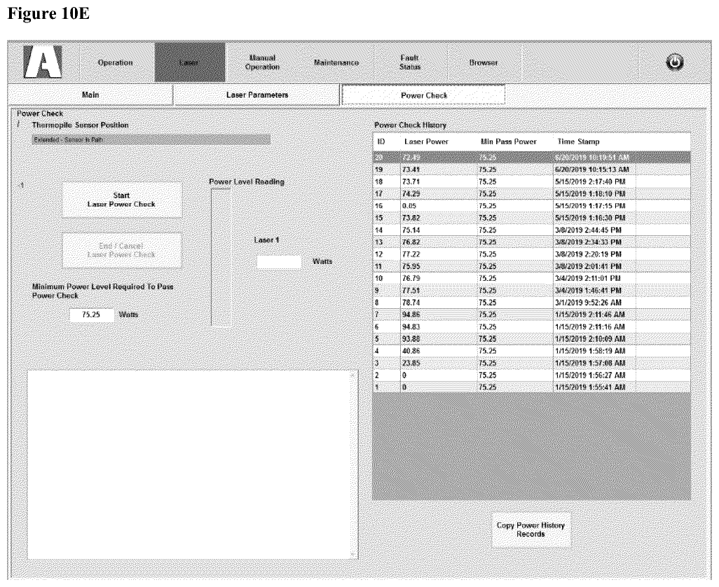

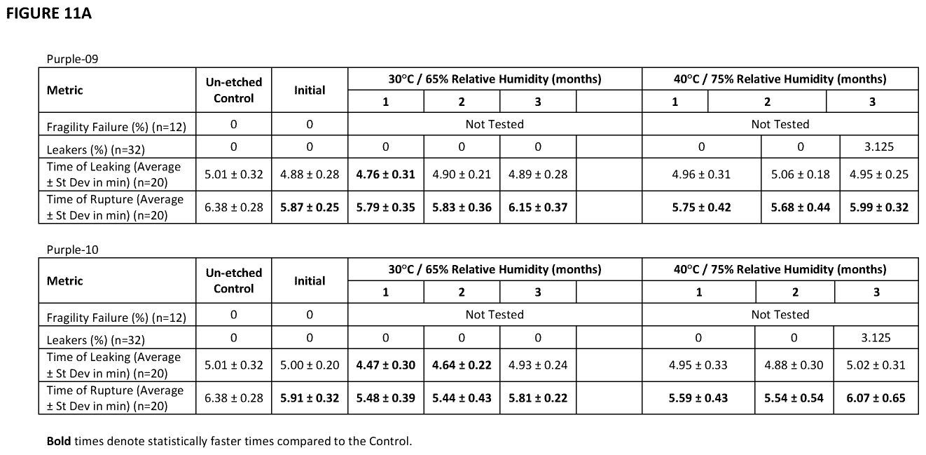

Referring to FIG. 10A, which show the main control screen when the system 100 is in operation. The software used to operate the system provides controls to select a product recipe (PRODUCT), select a paddle profile (PADDLE), activate the laser (FIRE LASER), toggle paddle pocket positions on or off, toggle the external fume extractor on or off, open or close the internal protective laser shutters, turn the visible diode guide on or off and save any changes made to the laser control boards (Apply Changes).

Referring to FIG. 10B, in the “Laser” screen, the “Image Parameters” tab allows selection of a pre-defined image for processing as well as a scaling factor to increase or decrease the size of the selected image. Referring to FIG. 10C, in the “Laser” screen, the “Laser Parameters” tab allows setting laser parameters specific for the PRODUCT selected including repetitions (Depth Count) and pause (Repeat Delay). Referring to FIG. 10D, in the “Laser” screen, the “Paddle Layout” tab allows positioning of the center of the laser processing point. Each logo etched can be individually positioned on each pocket in the paddle. Referring to FIG. 10E, in the “Laser” screen, the “Power Check” tab allows manual activation of a laser power check sequence which will provide the average wattage of the laser when continuously fired to ensure laser performance stability.

### Example 2

Light Amplification by Simulated Emission of Radiation (LASER) was utilized as highly focused light source to etch the surfaces of soft gelatin of capsules of various types. Some embodiments used a CO₂ laser designed for pharmaceutical use (Model 01811-00008, Ackley Machine Corporation, Moorestown, N.J.) and ScanMaster Controller settings (Cambridge Technology Company, Bedford, MA). Carbon monoxide lasers are also suitable for use according to the present disclosure, and a variety of laser types and models that are used for etching pharmaceutical products are known and are suitable for use according to the present disclosure.

Specific embodiments of ScanMaster of settings and processes used to etch the surface of opaque purple and red softgels are as follows, but other settings and durations can also be used as understood by a person of ordinary skill having the benefit of this disclosure.
Purple:
   DC1 100%, LP 100%, DC 4
   DC1 75%, LP 100%, DC 5
Red:
   DC1 100%, LP 100%, DC 4
   DC1 100%, LP 90%, DC 5

As used above: DC1—duty cycle percentage; LP—laser power percentage; DC—depth count (refers to the number of passes of the laser along the etching design). In addition, repeat delays of 0.5 and 1 second were found to be effective when etching the surface of softgels using the above settings. Longer or shorter delays can also be applied according to the present disclosure, although longer delays may increase costs when scaled for manufacturing large batches of softgels.

Additional embodiments used the settings provided in Table 2 below for opaque and red softgels. Other embodiments of settings used are provided where applicable.

| Batch | Purple-01 | Purple-02 | Red-01 | Red-02 |
|---|---|---|---|---|
| Quantity Etched | 500 | 500 | 500 | 500 |
| Etch Type | Double | Single ellipse | Double | Double |
| | ellipse | around single circle | ellipse | ellipse |
| Duty Cycle (%) | 100 | 100 | 100 | 100 |
| Mark Speed | 2000 | 2000 | 2000 | 2000 |
| (mm/s) | | | | |
| Laser Power (%) | 100 | 75 | 100 | 100 |
| Mark Speed (%) | 100 | 100 | 100 | 100 |
| Depth Count | 2 | 2 | 2 | 3 |
| Repeat Delay | 0.5 | 0.5 | 0.5 | 0.5 |

### Example 3

LASER was used to etch the surface of various softgels to create channels or furrows that penetrated partially into the surface of the softgels but did not perforate the capsules and reach the interior contents. The depth of the furrows varied between ^{˜}106 microns and ^{˜}290 microns depending on the type of softgel materials used and the number of etches.

Laser Etched Softgels—Depth Comparisons

A small batch of capsules (3 for each setting) were tested to determine the effect of lasing on the softgel surface and determine depth settings that would be effective generating softgels that have on their surface laser-etched furrows that remove a portion of the surface of the capsule, but that do not penetrate the capsule and expose the interior contents to the outside environment.

TABLE 3
Etching Depth Comparison

| | Intact Softgel Depth | | | 1^{st} Etch Depth | | | 2nd Etch Depth | | | 3^{rd} Etch Depth | | | 4^{th} Etch Depth | | |
|---|---|---|---|---|---|---|---|---|---|---|---|---|---|---|---|
| | (μm) | | | (μm) | | | (μm) | | | (μm) | | | (μm) | | |
| | Low | High | Av | Low | High | Av | Low | High | Av | Low | High | Av | Low | High | Av |
| Capsule A | 320 | 347 | 333.5 | 280 | 296 | 288 | 176 | 284 | 280 | 268 | 278 | 273 | 231 | 237 | 234 |
| Capsule B | 374 | 390 | 382 | 189 | 201 | 195 | 191 | 197 | 194 | 171 | 191 | 181 | 96 | 116 | 106 |

Etching of each softgel was tested using up to 4 separate passes of the laser to etch the surface of the softgel. In this embodiment, the 4^{th} etch has a lower depth than the 1st one. Two types of softgels, Gel A (an opaque softgel with high levels of gelatin) and Gel B (a transparent softgel with lower levels of gelatin that are commercially available in the form of Alka Seltzer Plus liquid softgels) were tested in the present embodiment.

TABLE 4
Depth Comparison in Percentages when compared to un-etched softgels

| | Intact Softgel Depth | | 1^{st} Etch Depth | | 2nd Etch Depth | | 3^{rd} Etch Depth | | 4^{th} Etch Depth | |
|---|---|---|---|---|---|---|---|---|---|---|
| | (μm) | | (μm) | | (μm) | | (μm) | | (μm) | |
| | Av | % Depth | Av | % Depth | Av | % Depth | Av | % Depth | Av | % Depth |
| Capsule A | 333.5 | 0 | 288 | 86.35 | 280 | 83.95 | 273 | 81.85 | 234 | 70.16 |
| Capsule B | 382 | 0 | 195 | 51.04 | 194 | 50.78 | 181 | 47.38 | 106 | 27.74 |

This table shows the percentage of etch depth on softgels with etching on the surface when compared to the un-etched softgels. Etching was performed at varying depths. This can be seen in the difference between 1^{st} and the 4^{th} etches.

TABLE 5
Comparison With Laser Marking

| | Intact Softgel Depth | | Laser Marking | | 1^{st} Etch Depth | |
|---|---|---|---|---|---|---|
| | (μm) | | (μm) | | (μm) | |
| | Av Depth | % Depth | Av Depth | % Depth | Av Depth | % Depth |
| Capsule A | 333.5 | 0 | 5 | 1.5 | 288 | 86.35 |
| Capsule B | 382 | 0 | 5 | 1.3 | 195 | 51.04 |

Laser was used according to prior techniques to mark the surface of softgel capsules, and the results were compared with laser etched capsules according to embodiments of the present disclosure.

As shown by the above table, where the laser marking depth averaged 5 microns, the depth of the markings constituted only 1.5% of the total depth at the point of marking. By comparison, certain embodiments of the present invention had etching depths of 86.35% and 51.04% respectively.

### Example 4

The inventors surprisingly discovered that the angle of incidence of the laser at the point of contact had a significant effect on the ability to achieve optimum furrow depth and rupture of the capsules during disintegration testing. The inventors found that angles at the etching site from about 100 to about 120 degrees off of normal could increase the rate of capsule rupture by up to about 40% faster than the control capsules, which contained furrows generated using a laser at an angle of about 90 degrees (or normal) to a tangent drawn at the etching site. In some embodiments, the laser has an angle of incidence that lies from about 5° to about 40° off normal at the point of contact of the laser on the surface of the capsule. In some embodiments, the laser has angle of incidence that lies from about 10° to about 35° off normal at the point of contact of the laser on the surface of the capsule.

### Example 5: Laser Etched Softgels—Release Testing

Laser-etched softgels were tested for release using Sotax DT50 disintegration apparatus. A maximum of 3 capsules were loaded per test, without sinker discs, into the tube basket to place the capsules into outward facing tubes and to allow for visual assessment of release. Release times were broken down by measuring:
Time of leak: point at which white precipitate from the fill is visually observed to be consistently escaping from the capsule.
Time of rupture: point at which a visible hole forms in the top surface of the capsule, such that either capsule fill can flow out of the hole or the inside of the capsule is visible.

Visual assessment was conducted in the presence of sufficient lighting to illuminate the disintegration baths and visualize release times. Larger batches than those used in Example 2 were tested to determine the effects of different variations of vertical and horizontal etching on the release rate.

Vertical Etching:

The inventors tested etching of softgel surfaces along the vertical axis of two different types of softgels (purple and red). For the purple softgels, the inventors found statistically significant reduction in leak and rupture time for some batches (Table 6).

TABLE 6
Vertical Etching of Softgels

| | Un-etched | | Purple 3 | | Purple 4 | | Purple 5 | | Purple 6 | |
|---|---|---|---|---|---|---|---|---|---|---|
| | Time of | Time of | Time of | Time of | Time of | Time of | Time of | Time of | Time of | Time of |
| | Leak | Rupture | Leak | Rupture | Leak | Rupture | Leak | Rupture | Leak | Rupture |
| Lot # | (min) | (min) | (min) | (min) | (min) | (min) | (min) | (min) | (min) | (min) |
| | 5.77 | 7.32 | 4.95 | 7.22 | 5.67 | 7.70 | 5.43 | 7.02 | 5.17 | 6.48 |
| | 6.22 | 7.78 | 5.18 | 6.82 | 5.53 | 7.22 | 5.58 | 6.62 | 4.95 | 7.05 |
| | 5.52 | 7.43 | 6.00 | 7.28 | 5.43 | 6.85 | 6.67 | 7.68 | 5.45 | 7.28 |
| | 5.42 | 7.42 | | | | | | | 5.02 | 7.58 |
| | 5.77 | 7.72 | | | | | | | 5.48 | 7.45 |
| | 5.68 | 7.58 | | | | | | | 5.58 | 7.15 |
| | 4.85 | 7.10 | | | | | | | 5.52 | 6.87 |
| | 5.22 | 7.25 | | | | | | | 4.03 | 5.95 |
| | 5.85 | 7.78 | | | | | | | 5.20 | 6.48 |
| | 5.72 | 7.60 | | | | | | | 4.92 | 7.22 |
| Average | 5.60 | 7.50 | 5.38 | 7.11 | 5.54 | 7.26 | 5.89 | 7.11 | 5.13 | 6.95 |
| StDev | 0.38 | 0.23 | 0.55 | 0.25 | 0.12 | 0.43 | 0.67 | 0.54 | 0.46 | 0.51 |

| | | | | | | | | |
|---|---|---|---|---|---|---|---|---|
| p-value | 0.2156 | 0.0156 | 0.4052 | 0.1064 | 0.1680 | 0.0412 | 0.0111 | 0.0032 |

For the red softgels, the inventors found that vertical etching designs did not yield consistent and statistically significant differences in release time when compared with un-etched controls.

Horizontal Etching:

In some embodiments using batches of purple softgels, the inventors found that elliptical etches along the horizontal axis of the capsule surfaces provided a consistent and significant reduction in time of leak and time of rupture when compared to un-etched controls (approximately 8% faster time of leak and between 7% and 20% faster time of rupture). In other testing, tablets with etching along a single horizontal surface yielded statistically equivalent time of leak (5:08±0:26 vs 5:05±0:26; p=0.8302) and slower time of rupture (6:59±0:28 vs. 5:56±0:30; p=0.0001) as compared to capsules with etching along two horizontal surfaces.

In some embodiments using batches of red softgels, the inventors found that elliptical etches along a single horizontal axis of the surface of capsules provided a consistent and significant reduction in time of leak and time of rupture when compared to un-etched controls. The inventors found no significant difference in the time of rupture between softgels with etches on one side versus those with etches on two sides. The single-sided horizontal etching design yielded faster time of leak (5:29±0:31 vs. 6:00±0:34; p=0.0449) and statistically equivalent time of rupture (7:13±0:35 vs. 6:56±0:41; p=0.3437) as compared to the double-sided horizontal design. See Table 7.

TABLE 7
Horizontal Etching Single-Sided vs. Double-Sided

| | | | Red 3 | | Red 4 | | Red 5 | | Red 6 | |
|---|---|---|---|---|---|---|---|---|---|---|
| | Un-etched | | (single-sided) | | (single-sided) | | (double-sided) | | (double-sided) | |
| | Time of | Time of | Time of | Time of | Time of | Time of | Time of | Time of | Time of | Time of |
| | Leak | Rupture | Leak | Rupture | Leak | Rupture | Leak | Rupture | Leak | Rupture |
| Lot # | (min) | (min) | (min) | (min) | (min) | (min) | (min) | (min) | (min) | (min) |
| | 6.28 | 7.72 | 6.03 | 7.57 | 5.43 | 7.42 | 5.58 | 6.42 | 5.18 | 6.18 |
| | 6.10 | 7.13 | 4.93 | 6.42 | 5.02 | 6.65 | 5.02 | 5.93 | 5.75 | 6.72 |
| | 6.78 | 7.62 | 5.53 | 7.47 | 4.78 | 6.00 | 6.12 | 6.50 | 6.32 | 7.12 |
| | 6.68 | 8.25 | 5.92 | 8.00 | 4.73 | 6.37 | 6.58 | 8.08 | 6.32 | 7.18 |
| | 6.43 | 8.72 | 5.52 | 7.55 | 5.47 | 7.03 | 6.32 | 7.47 | 5.98 | 6.92 |
| | 6.15 | 7.32 | 5.22 | 6.38 | 5.30 | 6.67 | 6.48 | 7.32 | 6.75 | 7.28 |
| | 6.63 | 7.87 | 6.03 | 7.65 | 5.50 | 6.67 | 6.73 | 7.52 | 5.48 | 6.27 |
| | 6.55 | 7.57 | 4.52 | 6.98 | 4.78 | 6.78 | 5.62 | 6.48 | 5.88 | 7.02 |
| | 6.95 | 7.68 | 5.27 | 6.52 | 4.58 | 5.27 | 5.43 | 6.40 | 6.05 | 6.78 |
| | 7.67 | 8.63 | 5.92 | 7.62 | 5.55 | 6.67 | 6.18 | 7.27 | 5.35 | 6.35 |
| Ave. | 6.62 | 7.85 | 5.49 | 7.22 | 5.12 | 6.55 | 6.01 | 6.94 | 5.91 | 6.78 |
| StDev | 0.46 | 0.53 | 0.51 | 0.59 | 0.37 | 0.58 | 0.56 | 0.68 | 0.48 | 0.40 |

| | | | | | | | | |
|---|---|---|---|---|---|---|---|---|
| p-value | 0.0000 | 0.0103 | 0.0000 | 0.0000 | 0.0076 | 0.0018 | 0.0016 | 0.0000 |

In view of the more consistent disintegration results seen with softgels having etching on the horizontal surfaces when compared to those having etching on the vertical surfaces, the inventors elected to focus on embodiments that have horizontal etches. Regardless, persons of ordinary skill having the benefit of the present disclosure would understand that varying the design, size, and depth of horizontal etches can consistently increase the disintegration rate of softgels when compared to those lacking the etches. Understanding that etching softgels on two separate surfaces will likely require additional manufacturing steps and add increased logistical requirements for softgel processing when compared to those with etching on a single surface, the inventors focused stability testing on softgels that have etching along a single horizontal surface. A person of ordinary skill having the benefit of the present disclosure would nevertheless understand that different variations of the disclosed embodiments (including, for example, etching on multiple sides, with different designs, or in different orientations) can achieve the stability seen with the specific embodiments disclosed herein.

### Example 6

TABLE 8
Lasing Parameters of Stability Testing
in Bottles for Purple Softgels

| Batch | Purple 7 | Purple 8 |
|---|---|---|
| Etch Type | Concentric ellipses | Single ellipse around |
| | | single circle |
| Duty Cycle (%) | 100 | 100 |
| Mark Speed | 2000 | 2000 |
| (mm/s) | | |
| Laser Power (%) | 100 | 75 |
| Mark Speed (%) | 100 | 100 |
| Depth Count | 2 | 2 |
| Repeat Delay | 0.5 | 0.5 |

In some embodiments, stability testing was performed on batches of purple softgels bearing two types of etching on a single horizontal surface (either two concentric ellipses or a single ellipse around a circle, with 500 samples per batch). See below in Table 8.

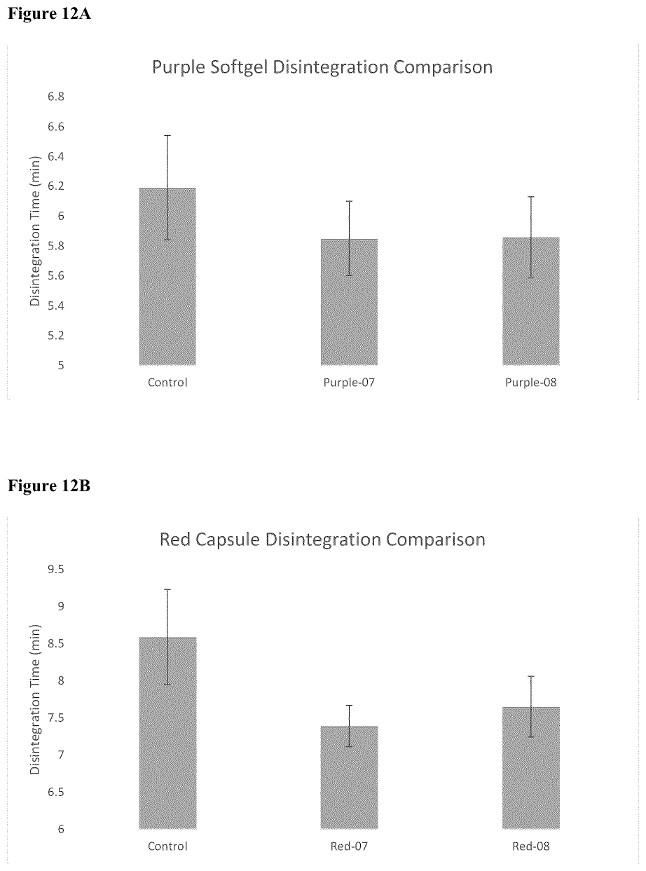

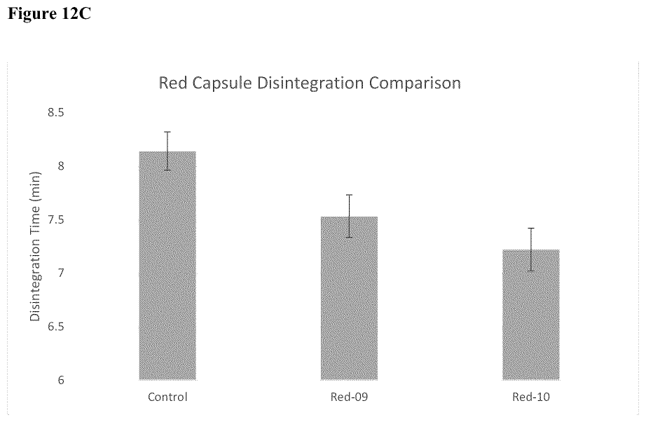

Disintegration testing showed a statistically significant increase in disintegration of the etched capsules when compared with controls as shown in FIG. 12A.

In addition, long term stability testing in amber jars (50 capsules per 8 oz jar) for up to 3 months was conducted to monitor physical characteristics, including leaking. See below in Table 9.

TABLE 9
Laser Etched Purple Softgels-3-Month
Stability Testing (% Leaking)*

| | Purple | Purple | Purple | Purple |
|---|---|---|---|---|
| Month | 07 @ RT | 07 @ 30° C. | 08 @ RT | 08 @ 30° C. |
| 0 | 0 | 0 | 0 | 0 |
| 1 | 0 | 2 | 0 | 2 |
| 2 | 0 | 0 | 2 | 0 |
| 3 | 0 | 0 | 6 | 2 |

*Under the testing protocol, a six percent failure rate is acceptable. No significant change in the trend of leak or rupture time was observed in the softgels over the course of the study.

TABLE 10
Lasing Parameters of Stability Testing in Bottles for Red Softgels

| Batch | Red-07 | Red-08 | Red-09 | Red-10 |
|---|---|---|---|---|
| Etch Type | Concentric | Concentric | Concentric | Concentric |
| | ellipses | ellipses | ellipses | ellipses |
| Duty Cycle (%) | 100 | 100 | 100 | 100 |
| Mark Speed | 4000 | 4000 | 2000 | 2000 |
| (mm/s) | | | | |
| Laser Power (%) | 100 | 90 | 100 | 100 |
| Mark Speed (%) | 100 | 100 | 100 | 100 |
| Depth Count | 4 | 5 | 2 | 3 |
| Repeat Delay | 0.5 | 0.5 | 0.5 | 0.5 |

In some embodiments, red softgels were tested for disintegration and long term stability in bottles after etching. The laser settings are provided below in Table 10.

Disintegration testing showed a significant difference in disintegration rates as compared to un-etched controls for batches Red-07 and Red-08 as shown in FIG. 12B.

In addition, long term stability testing in amber jars (50 capsules per 8 oz jar) for up to 3 months was conducted to monitor physical characteristics, including leaking. See below in Table 11.

TABLE 11
Laser Etched Red Softgels-3-Month Stability Testing (% Leaking)*

| | Red-07 | Red-07 | Red-08 | Red-08 |
|---|---|---|---|---|
| Month | @ RT | @ 30° C. | @ RT | @ 30° C. |
| 0 | 0 | 0 | 0 | 0 |
| 1 | 0 | 0 | 1 | 0 |
| 2 | 0 | 0 | 0 | 0 |
| 3 | 0 | 0 | 0 | 0 |

*Under the testing protocol, a six percent failure rate is acceptable. No significant change in the trend of leak or rupture time was observed in the softgels over the course of the study. Similar disintegration results were seen with batches Red-09 and Red-10 as shown in FIG 12C.

In addition, long term stability testing in amber jars (50 capsules per 8 oz jar) for up to 3 months was conducted to monitor physical characteristics, including leaking. See below in Table 12.

TABLE 12
Laser Etched Red Softgels-3-Month Stability Testing (% Leaking)*

| | Red-09 | Red-09 | Red-10 | Red-10 |
|---|---|---|---|---|
| Month | @ RT | @ 30° C. | @ RT | @ 30° C. |
| 0 | 0 | 0 | 0 | 0 |
| 1 | 0 | 0 | 0 | 0 |
| 2 | 0 | 0 | 0 | 0 |
| 3 | 0 | 0 | 0 | 0 |

*Under the testing protocol, a six percent failure rate is acceptable. No significant change in the trend of leak or rupture time was observed in the softgels over the course of the study.

### Example 7

TABLE 13
Laser Etched Softgels-Stability Testing in Blister Packs

| Batch | Purple-09 | Purple-10 | Red-09 | Red-10 |
|---|---|---|---|---|
| Etch Type | Concentric | Single ellipse | Concentric | Concentric |
| | ellipses | around single | ellipses | ellipses |
| | | circle | | |
| Duty Cycle (%) | 100 | 100 | 100 | 100 |
| Mark Speed | 2000 | 2000 | 2000 | 2000 |
| (mm/s) | | | | |
| Laser Power (%) | 100 | 60 | 100 | 75 |
| Mark Speed (%) | 100 | 100 | 100 | 100 |
| Depth Count | 2 | 2 | 2 | 3 |
| Repeat Delay | 0.5 | 0.5 | 0.5 | 0.5 |

In some embodiments, batches of softgel capsules (Purple and red capsules) were etched using the below lasing parameters:

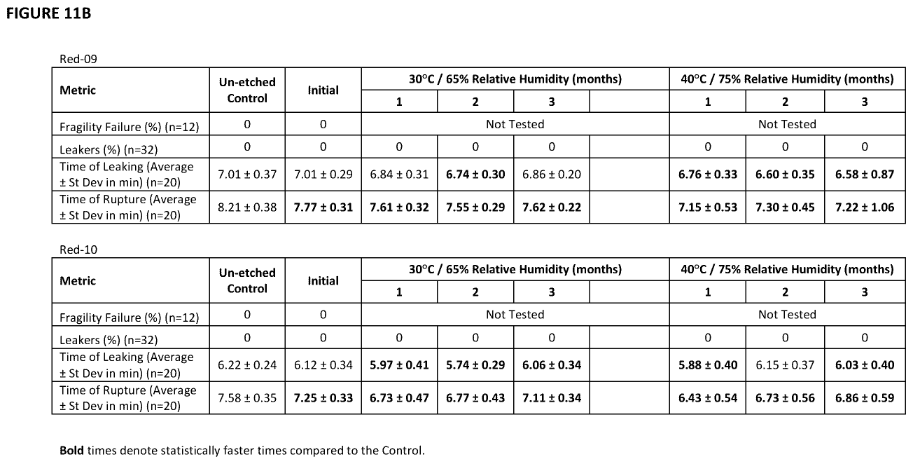

Samples from the above batches were stored in packs for blister capsules in the following conditions: 30° C./65% relative humidity and 40° C./75% relative humidity. The inventors found: 1) no fragility failure upon manufacture; 2) little to no leaking upon during exposure to stability conditions; 3) hatch-like opening during disintegration; 4) maintenance of faster rupture time when compared to non-etched controls. The maintenance of physical stability for 3 months at 40° C./75% RH is comparable to 2 years of shelf life at room temperature according to standard guidelines. The results appear in FIG. 11.

The present disclosure is not to be limited in terms of the particular embodiments or implementations described in this application, which are intended as illustrations of various aspects. Many modifications and embodiments can be made without departing from its spirit and scope. Functionally equivalent methods and apparatuses within the scope of the disclosure, in addition to those enumerated herein, are possible from the foregoing descriptions. Such modifications and embodiments are intended to fall within the scope of the appended claims. The present disclosure is to be limited only by the terms of the appended claims, along with the full scope of equivalents to which such claims are entitled. This disclosure is not limited to particular methods, which can, of course, vary. The terminology used herein is for the purpose of describing particular embodiments only and is not intended to be limiting.

With respect to the use of substantially any plural and/or singular terms herein, the terms can be translated from the plural to the singular and/or from the singular to the plural as is appropriate to the context and/or application. The various singular/plural permutations may be expressly set forth herein for sake of clarity.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, the disclosure is also thereby described in terms of any individual member or subgroup of members of the Markush group.

For any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. Language such as “up to,” “at least,” “greater than,” “less than,” and the like include the number recited and refer to ranges which can be subsequently broken down into subranges as discussed above. A range includes each individual member. Thus, for example, a group having 1-3 items refers to groups having 1, 2, or 3 items. Similarly, a group having 1-5 items refers to groups having 1, 2, 3, 4, or 5 items, and so forth.

While various aspects and embodiments have been disclosed herein, other aspects and embodiments are possible. The various aspects and embodiments disclosed herein are for purposes of illustration and are not intended to be limiting.

1. A softgel capsule comprising at least one laser etched channel positioned on an outer surface of the capsule, wherein the at least one channel penetrates at least partially into the surface of the capsule and reduces a structural integrity of the capsule, while preventing exposure of the interior contents of the capsule to the outside environment and facilitating release of the interior contents of the capsule, and wherein the at least one laser etched channel comprises a depth sufficient to facilitate disintegration of the capsule by increasing capsule surface area, facilitating erosion, and reducing capsule thickness at a site of the at least one laser etched channel.

2. (canceled)

3. (canceled)

4. (canceled)

5. (canceled)

6. (canceled)

7. (canceled)

8. The capsule of claim 1, wherein a depth of the at least one laser etched channel is from about 234 to about 288 microns.

9. The capsule of claim 1, wherein a depth of the at least one laser etched channel is from about 106 to about 105 microns.

10. The capsule of claim 1, wherein a depth of the at least one laser etched channel is from about 106 microns to about 290 microns.

11. (canceled)

12. A method of making an etched softgel capsule, the method comprising ablating a portion of an outer surface of the capsule with at least one laser beam to form at least one laser etching on the surface of the capsule, wherein the at least one laser etching penetrates at least partially into the surface of the capsule, and wherein the at least one laser etching provides a localized area of reduced thickness and reduced structural integrity as compared to a capsule free of laser etching.

13. (canceled)

14. (canceled)

15. (canceled)

16. The method of claim 12, wherein the at least one laser beam is positioned during the ablating at an angle that is greater than about 90° to a tangent drawn at a point of contact of the at least one laser beam on the surface of the capsule.

17. The method of claim 12, wherein the at least one laser beam has an angle of incidence that lies from about 5° to about 55° off normal at a point of contact of the at least one laser beam on the surface of the capsule.

18. (canceled)

19. (canceled)

20. (canceled)

21. (canceled)

22. (canceled)

23. The method of claim 12, wherein the capsule is a softgel, and wherein the at least one laser beam has an angle of incidence that lies from about 15° to about 35° off normal at a point of contact of the at least one laser beam on the surface of the capsule.

24. The method of claim 16, wherein the ablating comprises lasing the capsule for a first time period, pausing the ablating or a period from about 0.1 second to about 1 second, and resuming lasing of the capsule in the same position for a second time period.

25. (canceled)

26. (canceled)

27. The capsule of claim 1, wherein the capsule ruptures in the gastrointestinal tract at least about 10% faster than softgel capsules having a similar composition but that lack laser etching.

28. (canceled)

29. (canceled)

30. The capsule of claim 1, wherein a depth of the at least one laser etched channel is from about 70% to about 86% of an original depth of the softgel capsule at an etching site.

31. The capsule of claim 1, wherein a depth of the at least one laser etched channel is from about 27% to about 50% of an original depth of the softgel capsule at an etching site.

32. The capsule of claim 1, wherein a thickness of the capsule at the at least one laser etched channel is from about 30% to about 14% of an original capsule thickness at a site of the at least one laser etched channel.

33. The capsule of claim 1, wherein a thickness of the capsule at the at least one laser etched channel is from about 73% to about 50% of an original capsule thickness at a site of the at least one laser etched channel.

34. (canceled)

35. (canceled)

36. The capsule of claim 1, wherein a depth of the at least one laser etched channel is from about 106 microns to about 290 microns.

37. (canceled)

38. (canceled)

39. (canceled)

40. (canceled)

41. (canceled)

42. (canceled)

43. (canceled)

44. The softgel capsule of claim 1, wherein the at least one laser etched channel comprises multiple laser etched channels situated concentrically on the outer surface of the capsule.

45. (canceled)

46. (canceled)

47. (canceled)

48. The softgel capsule of claim 1, wherein the capsule is stable under ambient conditions for at least 12 months.

49. (canceled)

50. (canceled)

51. A system comprising:
(i) a controller;
(ii) a device comprising a laser and a processor; and
(iii) a non-transitory computer-readable storage medium in operable communication with the controller and the processor;
wherein the non-transitory computer-readable storage medium has instructions stored thereon for execution on the device and wherein the processor implements a method comprising:
(a) activating a laser positioned as a distance from a capsule;
(b) lasing a capsule disclosed herein along a first predetermined path for a time period sufficient to create an etching at a first predetermined depth;
(c) pausing the lasing from about 0.1 second to about 1 second;
(d) lasing the capsule disclosed herein along a second predetermined path for a time sufficient to create a second etching beginning at least partially at the first predetermined depth for a time period sufficient to create an etching at a second predetermined depth.

52. The system of claim 51, wherein the method further comprises repeating the pausing the lasing and the lasing the capsule until the etching is at a depth from about 106 to about 195 microns.

53. The system of claim 51, wherein the instructions of the non-transitory computer-readable storage medium further comprise one or a plurality of parameters that adjust the batch items of Table 13.

54. (canceled)
